(19) United States
(12) Patent Application Publication
(10) Pub. No.: US 2013/0323178 A1
(43) Pub. Date: Dec. 5, 2013

(54) INDOCYANINE GREEN-CONTAINING PARTICLE AND CONTRAST AGENT FOR PHOTOACOUSTIC IMAGING HAVING THE PARTICLE

(71) Applicant: CANON KABUSHIKI KAISHA, Tokyo (JP)

(72) Inventors: Fumio Yamauchi, Kyoto-shi (JP); Atsushi Takahashi, Kawasaki-shi (JP); Kengo Kanazaki, Kyoto-shi (JP); Satoshi Ogawa, Kyoto-shi (JP); Masato Minami, Kawasaki-shi (JP); Daisuke Sasaguri, Yokohama-shi (JP); Kouichi Kato, Yokohama-shi (JP); Yoshinori Tomida, Atsugi-shi (JP); Sachiko Yamauchi, Yokohama-shi (JP)

(73) Assignee: CANON KABUSHIKI KAISHA, Tokyo (JP)

(21) Appl. No.: 13/960,332

(22) Filed: Aug. 6, 2013

Related U.S. Application Data
(63) Continuation of application No. PCT/JP2013/001014, filed on Feb. 22, 2013.

(30) Foreign Application Priority Data
Feb. 23, 2012 (JP) 2012-038034
Jun. 21, 2012 (JP) 2012-139646
Nov. 30, 2012 (JP) 2012-263002

(51) Int. Cl. A61K 49/00; A61K 49/22

(52) U.S. Cl. CPC A61K 49/0093; A61K 49/0084; A61K 49/226; A61K 49/225
USPC 424/9.5; 424/9.6

(57) ABSTRACT

Provided is the following indocyanine green (ICG)-containing particle to be used as, for example, a contrast agent for fluorescent imaging or photoacoustic imaging. The leakage of ICG from the particle in a serum is prevented and hence the particle can stably retain ICG. According to a particle characterized by having an aggregate of indocyanine green and a phospholipid, the leakage of ICG from the particle in a serum or the like is prevented and hence ICG can be stably retained in the particle.

### CROSS-REFERENCE TO RELATED APPLICATIONS

This application is a continuation of International Application No. PCT/JP2013/001014, filed Feb. 22, 2013, which claims the benefit of Japanese Patent Application No. 2012-038034, filed Feb. 23, 2012, Japanese Patent Application No. 2012-139646, filed Jun. 21, 2012, and Japanese Patent Application No. 2012-263002, filed Nov. 30, 2012.

### BACKGROUND OF THE INVENTION

1. Field of the Invention

The present invention relates to a particle containing indocyanine green.

2. Description of the Related Art

In recent years, a fluorescence imaging or a photoacoustic imaging has attracted attention as an imaging method that allows non-invasive diagnosis.

The fluorescence imaging involves irradiating a fluorescent dye with light and detecting fluorescence emitted from the dye, and is widely used in various types of imaging. The photoacoustic imaging involves detecting an intensity and generation position of an acoustic wave resulting from volume expansion caused by heat released from a molecule as an object to be measured irradiated with light, to thereby obtain an image of the object to be measured. In the fluorescence imaging or photoacoustic imaging, a dye may be used as a contrast agent for increasing an intensity of fluorescence or an acoustic wave from a site to be measured.

In the contrast agent described above a dye that absorbs light to emit fluorescence or an acoustic wave is accumulated in, for example, a particle, a micelle, a polymer micelle, or a liposome (when the term “particle” is simply used, hereinafter, the term means a generic term for the foregoing unless otherwise stated) to increase a dye density. It may lead to an effective amplification of a signal intensity (intensity of fluorescence or an acoustic wave) and an improvement in absorption efficiency of irradiation energy.

Indocyanine green (sometimes abbreviated as ICG, hereinafter) is known as a dye known to emit fluorescence or an acoustic wave through light absorption. It should be noted that the ICG as used herein refers to a compound having a cyanine structure and having a structure represented by the following chemical formula 1.

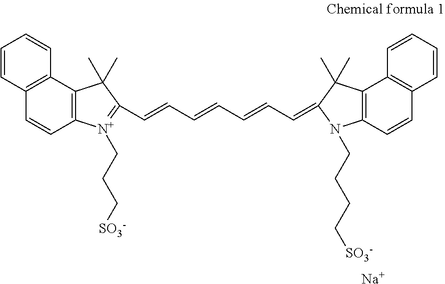

In this regard, H⁺ or K⁺ as well as Na⁺ may be used as the counter ion.

However, ICG has a low molecular weight, and hence is of a small size and has low retentivity in a lymph node. Consequently, a contrast agent for a lymph node having an additionally large size has been desired. In addition, an investigation that an additionally large amount of ICG is caused to reach a tumor site by encapsulating ICG in a particle has been conducted.

As a particle containing ICG and having an additionally large size, Journal of Photochemistry and Photobiology B: Biology, 74 (2004) 29-38 discloses an ICG-containing poly(lactide-co-glycolide) (hereinafter, sometimes abbreviated as PLGA) particle obtained by an emulsion solvent diffusion method using polyvinyl alcohol (PVA) as a surfactant.

### SUMMARY OF THE INVENTION

However, the particle containing ICG disclosed in Journal of Photochemistry and Photobiology B: Biology, 74 (2004) 29-38 involves a problem in that the ICG is a dye having a hydrophilic functional group and hence the ICG leaks out of the particle in an aqueous solution such as serum.

In a contrast agent containing a particle containing ICG, the ICG content of the particle in a living organism needs to be excellent. When the ICG content of the particle is low, the amount of ICG to be transported to a target tissue reduces and hence contrast sensitivity becomes insufficient. As a result, there arises a need for the administration of a large amount of ICG-containing particles. An ICG-containing particle having an excellent ICG content has been required to prevent a patient from receiving an excessive burden.

In view of the foregoing, an object of the present invention is to provide a particle capable of stably retaining a dye in itself in the case of administration into an aqueous solution such as a serum or to a mouse through the use of a J-aggregate of ICG as an embodiment of an aggregate of ICG.

In addition, a liposome encapsulating ICG described in Japanese Patent Application Laid-Open No. 2005-220045 involves the following problem. ICG leaks from a particle in an aqueous solution in a living organism or the like owing to a hydrophilic structure which ICG has. If ICG leaks in the living organism and hence the ICG content in the particle reduces, the amount of ICG to be transported to a tissue which one wishes to contrast reduces and hence contrast sensitivity becomes insufficient. As a result, there arises a need for the administration of a large amount of the ICG-encapsulating liposome. An ICG-encapsulating particle which suppresses the leakage of ICG in a living organism and has a high encapsulation ratio has been required to prevent a patient from receiving an excessive burden. In view of the foregoing, another object of the present invention is to provide an ICG-encapsulating particle having a high encapsulation ratio.

A particle according to the present invention is a particle, including: a J-aggregate of indocyanine green (ICG); and a lipid having a positively charged region.

An ICG-encapsulating particle according to the present invention is a particle including at least a phospholipid, cholesterol, and indocyanine green, in which a ratio of an absorbance of the particle at 700 nm to an absorbance thereof at 780 nm is 1 or more.

The particle according to the present invention can stably retain ICG in itself.

ICG hardly leaks from the particle according to the present invention.

Further features of the present invention will become apparent from the following description of exemplary embodiments with reference to the attached drawings.

### BRIEF DESCRIPTION OF THE DRAWINGS

FIG. 1 illustrates an example of a particle in an embodiment of the present invention.

FIG. 2 illustrates an example of another particle in the embodiment of the present invention.

FIG. 3 is the absorption spectrum of a contrast agent J-ICG-0.2 μm for a lymph node in Example 1. The absorption spectrum of ICG is superimposed for comparison.

FIG. 4A is the absorption spectra of an ICG-containing nanoparticle (ICG_NP1) and a J-aggregated ICG-containing nanoparticle (J-ICG_NP1) in Example 2.

FIG. 4B is the absorption spectra of an ICG-containing nanoparticle (ICG_NP2) and a J-aggregated ICG-containing nanoparticle (J-ICG_NP2) in Example 2.

FIG. 4C is the absorption spectra of an ICG-containing nanoparticle (ICG_NP3) and a J-aggregated ICG-containing nanoparticle (J-ICG_NP3) in Example 2.

FIG. 4D is the absorption spectrum of a J-aggregated ICG-containing nanoparticle (J-ICG_NP) in Example 2.

FIG. 5 is a graph showing the mean particle sizes of the various ICG-containing nanoparticles (ICG_NPs) and J-aggregated ICG-containing nanoparticles (J-ICG_NPs) produced by changing a DSPC loading amount in Example 2.

FIG. 6 is a graph showing the leakage ratios of the dyes of the various ICG-containing nanoparticles (ICG_NPs) and J-aggregated ICG-containing nanoparticles (J-ICG_NPs) produced by changing the DSPC loading amount in Example 2 into a serum.

FIG. 7 is an example of the absorption spectrum of a liposome JIL1-4C encapsulating a J-aggregate of ICG in Example 3.

FIG. 8A is an example of the in vivo fluorescence image of a cancer-bearing mouse to which the liposome JIL1-4C encapsulating the J-aggregate of ICG has been administered in Example 3, 24 hours after the administration.

FIG. 8B is an example of the in vivo fluorescence image of a cancer-bearing mouse to which ICG has been administered as a comparative example in Example 3, 24 hours after the administration.

FIG. 9 is a view showing the relative absorption spectrum (an absorption maximum in a measuring range is set to 1) and photoacoustic relative intensity (the highest photoacoustic intensity at a measurement wavelength is set to 1) of the J-ICG_NP1.

FIG. 10 is a schematic view of an ICG-encapsulating particle showing a 700/780 ratio of 1 or more in the present invention.

FIG. 11A is an example of the absorption spectrum of each of ICG-encapsulating particles prepared in Examples 7 and 8 of the present invention.

FIG. 11B is an example of the absorption spectrum of each of the ICG-encapsulating particles prepared in Examples 7 and 8 of the present invention.

FIG. 11C is an example of the absorption spectrum of each of the ICG-encapsulating particles prepared in Examples 7 and 8 of the present invention.

FIG. 12A is a transmission electron microscope observation image of an ICG-encapsulating particle PLD1 prepared in Example 7 of the present invention.

FIG. 12B is a transmission electron microscope observation image of an ICG-encapsulating particle C0 prepared in Example 7 of the present invention.

FIG. 13 is a view showing a relationship between the 700/780 ratio and retention ratio of ICG after 24 hours in a serum of each of the ICG-encapsulating particles prepared in Examples 7 and 8 of the present invention.

FIG. 14 is a view showing a relationship between the addition concentration of dextran 40 used in the preparation of the ICG-encapsulating particle prepared in Example 7 of the present invention and the 700/780 ratio.

FIG. 15 is an example of an image obtained by superimposing the bright-field image and fluorescence image of each of cancer-bearing mice to which the ICG-encapsulating particles PLD1 and EPLD1 prepared in Examples 7 and 8 of the present invention each showing a 700/780 ratio of 1 or more have been administered, and a cancer-bearing mouse to which ICG has been administered as a comparative example 24 hours after the administration.

FIG. 16 is a step flow chart of a particle size reduction treatment of the present invention.

FIG. 17A is a particle size distribution diagram based on cumulant analysis measured by a dynamic light scattering method (DLS method) in a main step of a particle size reduction treatment in Example 14 of the present invention.

FIG. 17B is a particle size distribution diagram based on the cumulant analysis measured by the dynamic light scattering method (DLS method) in the main step of the particle size reduction treatment in Example 14 of the present invention.

FIG. 17C is a particle size distribution diagram based on the cumulant analysis measured by the dynamic light scattering method (DLS method) in the main step of the particle size reduction treatment in Example 14 of the present invention.

FIG. 17D is a particle size distribution diagram based on the cumulant analysis measured by the dynamic light scattering method (DLS method) in the main step of the particle size reduction treatment in Example 14 of the present invention.

FIG. 18A is a particle size distribution diagram based on the cumulant analysis measured by the dynamic light scattering method (DLS method) in a main step of a particle size reduction treatment in Example 15 of the present invention.

FIG. 18B is a particle size distribution diagram based on the cumulant analysis measured by the dynamic light scattering method (DLS method) in the main step of the particle size reduction treatment in Example 15 of the present invention.

### DESCRIPTION OF THE EMBODIMENTS

Preferred embodiments of the present invention will now be described in detail in accordance with the accompanying drawings.

Hereinafter, embodiments of the present invention are described but the present invention is not limited to these embodiments.

A particle according to an embodiment of the present invention is characterized by having an aggregate of indocyanine green (ICG) and a phospholipid. ICG is known to form two kinds of aggregates as well as to exist as a monomer, and an H-aggregate formed of an aggregate based on a parallel fashion and a J-aggregate formed of an aggregate based on a head-to-tail fashion exist. The monomers of ICG aggregate to form the J-aggregate or the H-aggregate, thereby causing a reduction in hydrophilicity. As a result, ICG hardly leaks from the particle.

In addition, a contrast agent for photoacoustic imaging having such particle and a dispersion medium shows a large molar absorption coefficient and provides a photoacoustic signal because ICG hardly leaks from the particle.

Hereinafter, details about the embodiments of the present invention are described.

### Embodiment 1

An embodiment of the present invention relates to a particle containing the J-aggregate of indocyanine green (ICG).

(J-Aggregate)

ICG is known to form a J-aggregate under a specific condition (Chemical Physics, Volume 220, 1997, Pages 385-392, Chemical Physics, Volume 220, 1997, Pages 373-384 and Chemical Physics, Volume 269, 2001, Pages 399-409). The J-aggregate is a multimer having a mean particle size of several micrometers, and it is known that its absorption maximum wavelength largely moves to longer wavelengths and its absorption band becomes sharp as compared with a monomer.

The J-aggregate of ICG in this embodiment is defined as an aggregate having a local maximum of an absorbance between 880 nm and 910 nm as a result of the shift of an absorption wavelength out of the aggregates as the multimer structural products of ICG. It should be noted that when the J-aggregate of ICG is included in the particle in this embodiment, the monomer of ICG may be included in the particle. An abundance ratio between the J-aggregate and the monomer in the particle is not particularly limited as long as a ratio of the absorbance of the particle for light having a wavelength of 895 nm (derived from the J-aggregate) to its absorbance for light having a wavelength of 780 nm (derived from the monomer) is 0.1 or more. The ratio of the absorbance for light having a wavelength of 895 nm to the absorbance for light having a wavelength of 780 nm is preferably 1.0 or more, more preferably 2.0 or more, particularly preferably 5.0 or more. It should be noted that the J-aggregate of ICG may be used as a desalted body after having been treated with, for example, a desalting column.

In this embodiment, the use of the J-aggregate of ICG prevents the leakage of ICG from the particle to cause the particle to stably retain ICG in itself, and can improve the accumulation of ICG in a site to be measured such as a lymph node.

(Particle)

The particle of this embodiment is a particle containing the J-aggregate of ICG. The particle may contain an additive such as a lipid having a positively charged region as well as ICG, or may be a particle free of any additive. The particle may be, for example, a micelle, a polymer micelle, or a liposome as long as the J-aggregate of ICG is included so as to satisfy the absorption ratio. In addition, a surfactant may be present on the surface of the particle.

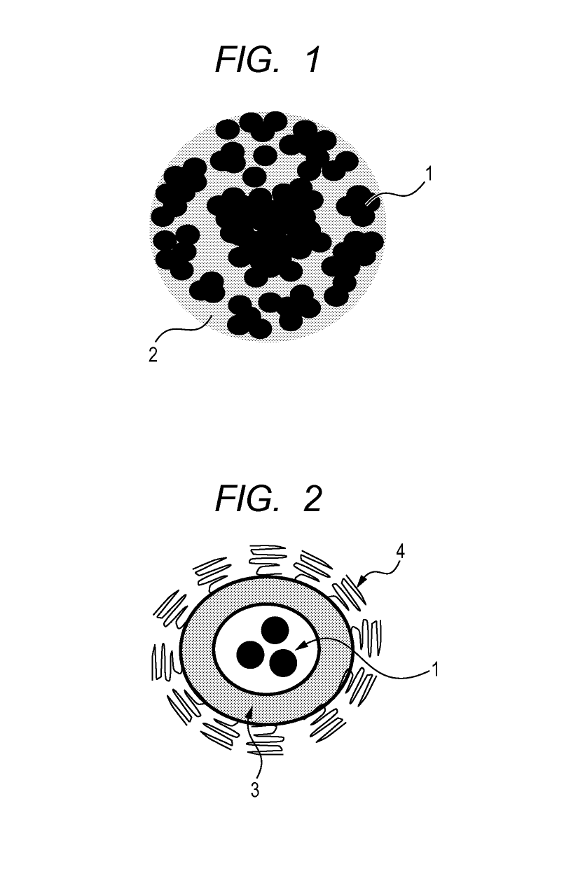

Examples of such particle include a particle formed only of the J-aggregate of ICG, such a particle formed of a J-aggregate 1 of ICG and a phospholipid 2 as an additive as illustrated in FIG. 1, and such a particle containing a surfactant 4 on the surface of a liposome 3 containing the J-aggregate 1 of ICG as illustrated in FIG. 2.

(Particle Size)

The particle size of the particle according to this embodiment is not particularly limited, provided that when the particle is used as a contrast agent, in particular, a contrast agent for a lymph node, setting its hydrodynamic mean particle size to 1,000 nm or less can enhance the ease with which the particle is taken in a lymph duct or a tissue (tissue permeability) and its retentivity in a lymph node or the tissue.

When the particle size is 1,000 nm or less, a larger amount of particles can be accumulated in a tumor site than that in a normal site in a living organism by an enhanced permeability and retention (EPR) effect. The tumor site can be specifically imaged by detecting the accumulated particles with various image-forming modalities such as fluorescence and photoacoustics. In addition, when the particle size exceeds 1,000 nm, efficient intake in a tissue such as a lymph duct cannot be expected. Consequently, the particle size is preferably set to 10 nm or more and 1,000 nm or less. The particle size is more preferably 20 nm or more and 500 nm or less, still more preferably 20 nm or more and 200 nm or less. This is because when the particle size of the particle is 200 nm or less, the particle is hardly taken in a macrophage in blood and hence its retentivity in the blood may improve.

The particle size can be measured through observation with an electron microscope or by a particle size-measuring method based on a dynamic light scattering method. When the particle size is measured based on the dynamic light scattering method, a hydrodynamic diameter is measured with a dynamic light scattering analyzer (DLS-8000, manufactured by Otsuka Electronics Co., Ltd.) by the dynamic light scattering (DLS) method.

As described in the foregoing, the J-aggregate of ICG has a particle size of several micrometers. Consequently, according to conventional findings, it has been impossible to expect efficient intake of the J-aggregate of ICG in a lymph duct. However, as demonstrated in an example according to this embodiment, a particle having such preferred size can be produced by filtering the aggregate with a pore filter to be described later, by employing a nanoemulsion method to be described later, or by incorporating the aggregate into a particle such as a liposome.

(Size Control with Pore Filter)

As described in a prior literature, the J-aggregate of ICG is a giant particle of several microns, specifically, an extremely polydisperse giant particle having a size of 3 microns in terms of a mean particle size. It has been elucidated in the example according to this embodiment that a particle of about 1,000 nm can be obtained by filtering a solution of the J-aggregate of ICG with a pore filter having a pore size of 1.2 μm. However, when the solution is filtered with the pore filter having a pore size of 1.2 μm, the polydispersity index of the J-aggregate of ICG is about 0.4, which means that its dispersibility is poor. Therefore, the particle in this embodiment is filtered with a pore filter having a pore size of preferably 0.45 μm or less, more preferably 0.2 μm or less from the viewpoint of tissue permeability when the particle is used as a contrast agent for a lymph node. When the particle in this embodiment is filtered with the pore filter having a pore size of 0.2 μm or less, the size of the particle can be reduced to about 300 nm. The particle size distribution of the particles filtered with the pore filter having a pore size of 0.2 μm or less becomes relatively narrow and the polydispersity index can be reduced to about 0.2. The pore filter is not particularly limited as long as the filter is a membrane filter having a predetermined pore size, and the filtration can be performed with a syringe filter or an extruder. A membrane filter of, for example, a cellulose or polycarbonate type can be appropriately used as the membrane filter, and its pore size desirably falls within the range of 0.05 to 0.4 μm, preferably 0.1 to 0.22 μm.

(Additive)

An example of the additive which the particle according to this embodiment may contain is a lipid having a positively charged region. Although ICG is a hydrophilic dye having a sulfonic group as a hydrophilic site, the addition of the lipid having a positively charged region results in the aggregation of the positively charged region which such additive has to the hydrophilic site of ICG, and hence can improve the hydrophobicity of ICG (the J-aggregate of ICG in this embodiment). Consequently, it is assumed that ICG can be solubilized in an organic solvent such as chloroform or dichloromethane.

(Lipid Having Positively Charged Region)

The lipid having a positively charged region is a lipid having a partial structure of a cation in a part of its structure. Examples of such lipid may include: glycerolipids such as a phosphatidylcholine, a phosphatidylethanolamine, and a phosphatidylserine; sphingolipids such as a sphingomyelin, a sphingophospholipid, and sphingosine; a glycolipid such as a sphingoglycolipid having an aminosugar moiety such as neuraminic acid; synthetic cholesterols such as cholesteryl-3β-carboxyamidoethylene-N-hydroxyethylamine and 3-([N—N′,N′-dimethylaminoethane)-carbamoyl]cholesterol; synthetic lipids such as laurylamine, stearylamine, N-[1-(2,3-dioleyloxy)propyl]-N,N,N-trimethylammonium chloride (abbreviation: DOTMA), and 2,3-dioleyloxy-N-[2-(sperminecarboxyamido)ethyl]-N,N-dimethyl-1-propanaminium trifluoroacetate (abbreviation: DOSPA); and an ether-type phospholipid and a cationic lipid.

In addition, examples of the phosphatidylcholine, phosphatidylethanolamine, and phosphatidylserine include diacylphosphatidylcholine, diacylphosphatidylethanolamine, and diacylphosphatidylserine.

In addition, the lipid having a positively charged region is preferably further having a phosphodiester bond. Examples thereof include 1,2-distearoyl-sn-glycero-3-phosphoethanolamine (DSPE), 1,2-dipalmitoyl-sn-glycero-3-phosphoethanolamine (DPPE), 1,2-dimyristoyl-sn-glycero-3-phosphoethanolamine (DMPE), 1,2-dilauroyl-sn-glycero-3-phosphoethanolamine (DLPE), 1,2-dioleoyl-sn-glycero-3-phosphoethanolamine (DOPE), 1,2-dilinoleoyl-sn-glycero-3-phosphoethanolamine (DLoPE), 1,2-dierucoyl-sn-glycero-3-phosphoethanolamine (DEPE), 1,2-distearoyl-sn-glycero-3-phospho-L-serine (DSPS), 1,2-dipalmitoyl-sn-glycero-3-phospho-L-serine (DPPS), 1,2-dimyristoyl-sn-glycero-3-phospho-L-serine (DMPS), 1,2-dioleoyl-sn-glycero-3-phospho-L-serine (DOPS), 1,2-distearoyl-sn-glycero-3-phosphocholine (DSPC), 1,2-dipalmitoyl-sn-glycero-3-phosphocholine (DPPC), 1,2-dimyristoyl-sn-glycero-3-phosphocholine (DMPC), 1,2-dilauroyl-sn-glycero-3-phosphocholine (DLPC), 1,2-dioleoyl-sn-glycero-3-phosphocholine (DOPC), and 1,2-dilinoleoyl-sn-glycero-3-phosphocholine (DLoPC).

As the lipid having a positively charged region in this embodiment, there may also be used, for example, 1,2-di-o-acyl-sn-glycero-3-phosphocholine, 1,2-diacyl-3-trimethylammonium propane chloride, o,o′-ditetradecanoyl-N-(α-trimethylammonioacetyl)diethanolamine chloride, and a hydrogenated soy phosphatidylcholine (sometimes abbreviated as HSPC).

As described later in examples, the use of a phospholipid at the time of the preparation of the particle in this embodiment can control the size of the particle. In addition, the surface characteristics of the particle can be changed by causing the phospholipid to adsorb to the surface of the J-aggregate of ICG. For example, a polyethylene glycol (PEG) can be introduced into the surface of the particle by causing a pegylated phospholipid to adsorb to the surface of the J-aggregate of ICG. In addition, a particle having a controlled surface potential can be obtained by using a charged phospholipid.

The lipid having a positively charged region according to this embodiment is particularly preferably at least one of dioleyl phosphatidyl ethanolamine and distearoyl phosphatidylcholine.

(Liposome)

The liposome in this embodiment means a monolayer liposome and a multilayer liposome constituted of a lipid, a glycolipid, a phospholipid, a sterol, and a combination thereof. The liposome may be constituted of a mixture of different lipids, and a derivative of a lipid such as a polyethylene glycol-bonded phospholipid can be used. A conventionally known method can be employed as a method of preparing the liposome and a liposome having desired physical properties can be obtained by appropriately selecting a method. The kind, amount, and the like of a lipid can be appropriately selected according to the applications of the liposome. The particle size and surface potential of the liposome can be controlled by taking, for example, the amount and ratio of a lipid, and the charge of the lipid into consideration.

Preferred examples of the neutral phospholipid in the liposome of this embodiment include a soy or yolk lecithin, a lysolecithin, and a derivative of a hydrogenated product or hydroxide thereof. In addition, examples thereof may also include a semisynthetic phosphatidylcholine, a phosphatidylserine (PS), a phosphatidylethanolamine, a phosphatidylglycerol (PG), a phosphatidylinositol (PI), and a sphingomyelin. In addition, an alkyl or alkenyl derivative of synthetic phosphatidic acid (PA) or the like may also be used, and examples thereof include distearoylphosphatidylcholine (DSPC), dimyristoylphosphatidylcholine (DMPC), dipalmitoylphosphatidylcholine (DPPC), dioleylphosphatidylcholine (DOPC), distearoylphosphatidylserine (DSPS), distearoylphosphatidylglycerol (DSPG), and dipalmitoylphosphatidic acid (DPPA).

Examples of the glycolipid include: a glycerolipid such as digalactosyldiglyceride; and sphingoglycolipids such as a galactosylceramide and a ganglioside.

Any other molecule may be added as a constituent molecule of liposome membrane other than a lipid as required. Examples thereof include: a cholesterol acting as a membrane stabilizing agent; a glycol such as ethylene glycol; a saccharide such as dextran; and a phosphoric acid dialkyl ester added to control a charge; and an aliphatic amine such as stearylamine.

(Encapsulation of ICG in Liposome)

The phrase “particle contains ICG” in this embodiment comprehends the case where ICG is encapsulated in the liposome. ICG (the J-aggregate of ICG in this embodiment) to be encapsulated in the liposome is a water-soluble substance and is typically encapsulated in the internal aqueous phase of the liposome. However, ICG has an affinity for a phospholipid and the multimerization of ICG molecules is liable to occur, and hence its localization to the surface of a liposome membrane or in a lipid bilayer membrane can occur. In this embodiment, the three cases, i.e., “encapsulation in the internal aqueous phase of the liposome,” “localization in the liposome membrane,” and “localization to the surface of the liposome” are collectively referred to as “encapsulation.”

(Surfactant)

The surfactant according to this embodiment is not particularly limited and may be any surfactant as long as it can form a particle emulsion. For example, a nonionic surfactant, an anionic surfactant, a cationic surfactant, a polymeric surfactant, and a phospholipid may be used. One kind of those surfactants may be used alone, or two or more kinds thereof may be used in combination. Examples of the nonionic surfactant to be used in the surfactant in this embodiment may include: polyoxyethylene sorbitan-based fatty acid esters such as Tween 20, Tween 40, Tween 60, Tween 80, and Tween 85; and Brij 35, Brij 58, Brij 76, Brij 98, Triton X-100, Triton X-114, Triton X-305, Triton N-101, Nonidet P-40, Igepol CO530, Igepol CO630, Igepol CO720, and Igepol CO730.

In addition, examples of the anionic surfactant to be used in the surfactant in this embodiment may include: sodium dodecyl sulfate; and a dodecylbenzenesulfonate, a decylbenzenesulfonate, an undecylbenzenesulfonate, a tridecylbenzenesulfonate, and a nonylbenzenesulfonate, and sodium, potassium, and ammonium salts thereof.

In addition, examples of the cationic surfactant to be used in the surfactant in this embodiment may include cetyltrimethylammonium bromide, hexadecylpyridinium chloride, dodecyltrimethylammonium chloride, and hexadecyltrimethylammonium chloride.

In addition, examples of the polymeric surfactant to be used in the surfactant in this embodiment may include polyvinyl alcohol, polyoxyethylene polyoxypropylene glycol, and gelatin. As a commercially available product of polyoxyethylene polyoxypropylene glycol, for example, Pluronic F68 (manufactured by Sigma-Aldrich Japan K.K.) and Pluronic F127 (manufactured by Sigma-Aldrich Japan K.K.) are given.

In addition, the phospholipid to be used in the surfactant in this embodiment is preferably a phosphatidyl-based phospholipid having a functional group selected from a hydroxyl group, a methoxy group, an amino group, a carboxyl group, an N-hydroxysuccinimide group, and a maleimide group. In addition, the phospholipid to be used in the surfactant may contain a PEG chain.

In order that the EPR effect proposed as a principle of passive targeting to a tumor may be caused, a contrast agent is required to have high retentivity in blood. The introduction of a polyethylene glycol into the particle of this embodiment is extremely useful because of the reason that: when its interaction with a protein in blood is suppressed, the polyethylene glycol is hardly phagocytosed by a reticuloendothelial cell of a liver or the like and hence can improve the retentivity of the particle in the blood.

A function of the polyethylene glycol can be regulated by appropriately changing its molecular weight and its ratio of introduction into the particle. A polyethylene glycol having a molecular weight of 500 to 200,000 is preferably used and the molecular weight is particularly suitably 2,000 to 100,000. In addition, the ratio of introduction of the polyethylene glycol into the particle is 0.001 to 50 mol %, preferably 0.01 to 30 mol %, more preferably 0.1 to 10 mol % with respect to the lipid constituting the particle.

Any known technique can be used as a method of introducing a polyethylene glycol into the particle. Preferred examples thereof include a method involving incorporating a polyethylene glycol-bonded phospholipid or the like into a phospholipid to be used for covering the particle in advance to produce the particle. Examples of the polyethylene glycol-bonded phospholipid include a polyethylene glycol derivative of a phosphatidylethanolamine, such as a distearoylphosphatidylethanolamine-polyethylene glycol (DSPE-PEG).

As a phospholipid to be used for a surfactant containing a PEG chain and a functional group such as a hydroxy group, a methoxy group, an amino group, an N-hydroxysuccinimide group, or a maleimide group, there may be given, for example, phospholipids such as: 1,2-distearoyl-sn-glycero-3-phosphoethanolamine-N-[poly(ethylene glycol)] (DSPE-PEG-OH) represented by the chemical formula 2; poly(oxy-1,2-ethanediyl), α-[7-hydroxy-7-oxido-13-oxo-10-[(1-oxooctadecyl)oxy]-6,8,12-trioxa-3-aza-7-phosphatriacont-1-yl]-ω-methoxy- (DSPE-PEG-OMe) represented by the chemical formula 3; N-(aminopropyl polyethyleneglycol)-carbamyl distearoylphosphatidyl-ethanolamine (DSPE-PEG-NH2) represented by the chemical formula 4; 3-(N-succinimidyloxyglutaryl)aminopropyl polyethyleneglycol-carbamyl distearoylphosphatidyl-ethanolamine (DSPE-PEG-NHS) represented by the chemical formula 5; and N-(3-maleimide-1-oxopropyl)aminopropyl polyethyleneglycol-carbamyl distearoylphosphatidyl-ethanolamine (DSPE-PEG-MAL) represented by the chemical formula 6. It should be noted that in the chemical formulae 2 to 6, n represents an integer of 5 or more and 500 or less.

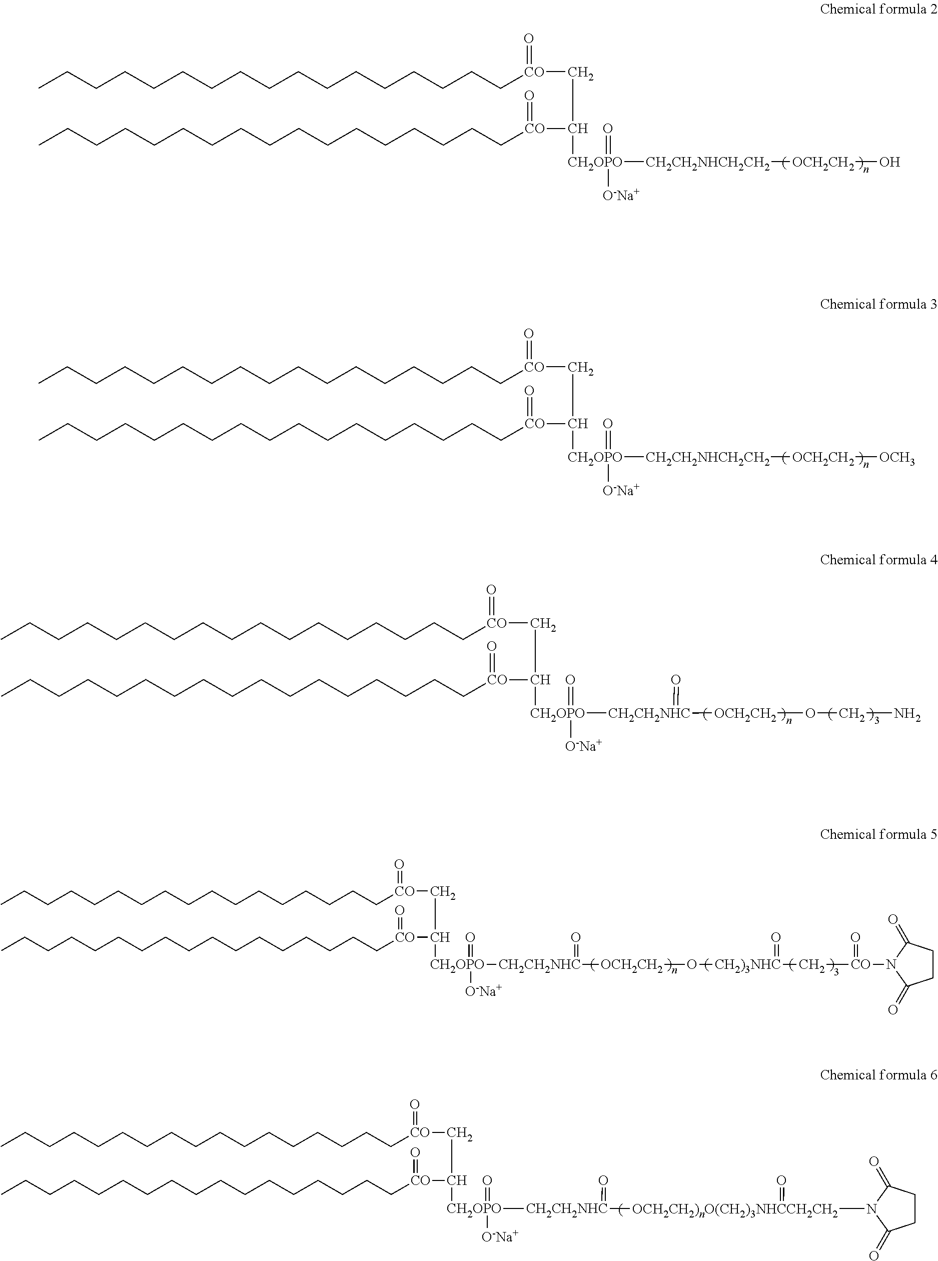

It should be noted that the number of kinds of the surfactants to be used in this embodiment is not limited to one, and two or more kinds of surfactants may be simultaneously used.

(Targeting Molecule)

In this embodiment, a target site can be specifically labeled by immobilizing a targeting molecule to part of the particle.

The targeting molecule is, for example, a substance that specifically binds to a target site such as a tumor or a substance that specifically binds to a substance present around the target site, and can be arbitrarily selected from, for example, chemical substances such as a biomolecule and a drug. Specific examples thereof include an antibody, an antibody fragment, an enzyme, a bioactive peptide, a glycopeptide, a sugar chain, a lipid, and a molecule-recognizing compound. One kind of those substances can be used alone, or two or more kinds thereof can be used in combination.

The use of the particle to which the targeting molecule is chemically bonded enables specific detection of the target site, and the tracking of the movement, localization, drug effect, metabolism, and the like of a target substance. For example, the adoption of a substance that specifically binds to a tumor as the targeting molecule enables specific detection of the tumor. In addition, the use of a substance, which specifically binds to a biological substance such as a protein or an enzyme present in a large amount around a specific disease site, as the targeting molecule enables specific detection of the disease. It should be noted that according to the particle according to this embodiment, the tumor can be detected by the EPR effect even when the particle has no targeting molecule.

(Immobilization of Targeting Molecule)

Any one of the known methods can be employed as a method of immobilizing the targeting molecule to the containing particle as long as the targeting molecule can be chemically bonded to the particle, though any known method varies depending on the kind of the targeting molecule to be used. For example, a method involving causing a functional group which the surfactant has and a functional group of the targeting molecule to react with each other to chemically bond the targeting molecule can be employed.

For example, when the surfactant is a phosphatidyl-based phospholipid having an N-hydroxysuccinimide group, the surfactant is caused to react with a targeting molecule having an amino group, thus the targeting molecule can be immobilized to the particle. After immobilization of the targeting molecule, it is preferred that unreacted N-hydroxysuccinimide groups of the surfactant be inactivated by causing to react with glycine, ethanolamine, an oligoethylene glycol or polyethylene glycol having an amino group at its terminal, or the like.

When the surfactant is a phosphatidyl-based phospholipid having a maleimide group, the surfactant is caused to react with a targeting molecule having a thiol group, thus the targeting molecule can be immobilized to the particle. After immobilization of the targeting molecule, it is preferred that unreacted maleimide groups of the surfactant be inactivated by causing to react with L-cysteine, mercaptoethanol, an oligoethylene glycol or polyethylene glycol having a thiol group at its terminal, or the like.

When the surfactant is a phosphatidyl-based phospholipid having an amino group, the surfactant is caused to react with an amino group of a targeting molecule through use of glutaraldehyde, thus the targeting molecule can be immobilized on the particle. After immobilization of the targeting molecule, it is preferred that the activity of unreacted amino groups be blocked by causing to react with ethanolamine, an oligoethylene glycol or polyethylene glycol having an amino group at its terminal, or the like. Alternatively, a targeting molecule can be immobilized through substitution of amino group of the surfactant with an N-hydroxysuccinimide group or a maleimide group.

(Method of Producing Particle)

A method of producing the particle according to this embodiment includes the steps of: transforming ICG into a J-aggregate; and preparing a particle containing ICG. The order of those steps does not matter as long as the particle of this embodiment is obtained.

(Step of Transforming ICG into J-Aggregate)

ICG can be transformed into a J-aggregate by, for example, the following “method (1),” “method (2),” or “method (3),” though a method for the transformation is not limited thereto.

Re: Method (1)

An aqueous solution of ICG (ICG concentration=1.5 mM) is warmed in a dark place at 65° C. for 32 hours. Next, the ICG solution is stored at room temperature for 5 days in a dark place to form a stable J-aggregate of ICG (Chemical Physics, Volume 220, 1997, Pages 385-392, Chemical Physics, Volume 220, 1997, Pages 373-384 and Chemical Physics, Volume 269, 2001, Pages 399-409). The J-aggregate is a multimer having a mean particle size of several micrometers and has a local maximum of an absorbance between 880 nm and 910 nm. The step of transforming ICG into a J-aggregate may be performed at any one of the following timings: before and after the preparation of a particle containing ICG, and in the midst of the preparation of the particle. Particles having a mean particle size of several hundreds of nanometers can be obtained by filtering the solution containing the J-aggregate of ICG with a pore filter.

Re: Method (2)

Particles each containing a J-aggregate of ICG can be obtained by warming ICG-containing particles obtained by, for example, the nanoemulsion method to be described later at 37° C. for 12 hours. The J-aggregate-containing particles thus obtained have a mean particle size of 200 nm or less and each have a local maximum of an absorbance between 880 nm and 910 nm.

Re: Method (3)

Particles each containing a J-aggregate of ICG can be obtained through the encapsulation of ICG in a liposome by a pH-gradient method to be described later. The J-aggregate-containing particles thus obtained have a mean particle size of about 100 nm and each have a local maximum of an absorbance between 880 nm and 910 nm.

(Step of Preparing Particle Containing ICG)

A particle containing ICG can also be prepared by a known method.

Hereinafter, a nonlimitative example of the method of producing the particle according to this embodiment is given.

(Production Example Involving Filtering J-Aggregate of ICG with Pore Filter)

First, an aqueous solution of ICG (ICG concentration=1.5 mM) is warmed in a dark place at 65° C. for 24 hours. Next, the ICG solution is stored at room temperature for 5 days in a dark place to form a stable J-aggregate of ICG. Next, as elucidated in the example according to this embodiment, a particle of about 300 nm can be prepared by filtering the aqueous solution of the J-aggregate of ICG with a pore filter having a pore size of 0.1 μm and recovering the filtrate. It should be noted that the particle may be prepared by adding a phospholipid such as HSPC or DSPC at the time of the warming of the aqueous solution of ICG so that its concentration may be 3 mM, or the particle may be prepared by adding the phospholipid after transforming ICG into the J-aggregate. The particle obtained in this embodiment is characterized by having a particle size of several hundreds of nanometers which has been impossible to obtain by a conventional production method. In particular, a particle obtained by adding HSPC at the time of the warming of the aqueous solution of ICG is a particle of about 30 nm containing the J-aggregate of ICG, and in the production method of this embodiment, the particle size can be reduced to a level of several tens of nanometers.

(Production Example of Particle Containing Additive)

A particle containing an additive can be prepared by, for example, the nanoemulsion method. An example of the steps of production by the nanoemulsion method is as described below.

Specifically, an aqueous dispersion liquid of a particle containing ICG can be obtained by the following steps (A) to (C):

(A) the step of adding a first liquid obtained by dissolving ICG and the additive in an organic solvent to a second liquid as an aqueous solution prepared by dissolving a surfactant to provide a mixed liquid;
(B) the step of emulsifying the mixed liquid to provide an O/W type emulsion; and
(C) the step of distilling off the first liquid from the dispersoid of the emulsion.

When two or more kinds of surfactants are used, or when no surfactant is used, an aqueous solution having arbitrary composition has only to be used as the second liquid in the step (A).

(First Liquid)

Any organic solvent can be used as the organic solvent to be used as a solvent for the first liquid to be used in the nanoemulsion method as long as the solvent has no solubility or small solubility in water, and can dissolve a composition formed of ICG and the additive, provided that the solvent is preferably a volatile organic solvent.

Such organic solvent is not limited, and examples thereof include halogenated hydrocarbons (such as dichloromethane, chloroform, chloroethane, dichloroethane, trichloroethane, and carbon tetrachloride), ethers (such as ethyl ether and isobutyl ether), esters (such as ethyl acetate and butyl acetate), and aromatic hydrocarbons (such as benzene, toluene, and xylene). These organic solvents may be used alone, or two or more kinds thereof may be used by mixing at an appropriate ratio.

The concentration of ICG in the first liquid is preferably set to 0.0005 to 100 mg/ml.

The weight ratio between ICG and an additive in the first liquid preferably falls within the range of 10:1 to 1:20.

(Second Liquid)

The second liquid to be used in the nanoemulsion method is an aqueous solution, more preferably an aqueous solution prepared by dissolving the surfactant. When the surfactant is incorporated into the second liquid in advance, the emulsion can be stabilized upon mixing of the liquid with the first liquid, provided that in this embodiment, it is sufficient that the surfactant can be incorporated into a dispersion liquid prepared by mixing the first liquid and the second liquid, and the surfactant is not necessarily needed to be dissolved in the second liquid in advance.

A preferred concentration of the surfactant in the second liquid varies depending on the kind of the surfactant to be used, and a mixing ratio between the first liquid and the second liquid. For example, when a nonionic surfactant, an anionic surfactant, a cationic surfactant, or a polymeric surfactant is used, the concentration in the second liquid is preferably set to 0.1 mg/ml to 100 mg/ml. In addition, for example, when a phospholipid containing a PEG chain is used as the surfactant, the concentration in the second liquid is preferably set to 0.001 mg/ml to 100 mg/ml.

When two kinds of surfactants (a surfactant A and a surfactant B) are used, a construction ratio between the surfactant A and the surfactant B in the case where a nonionic surfactant, an anionic surfactant, a cationic surfactant, or a polymeric surfactant is used as the surfactant A and a phospholipid containing a PEG chain is used as the surfactant B preferably falls within the range of 100:1 to 1:1 in terms of a molar ratio. A construction ratio between the surfactant A and the surfactant B outstripping the range is not preferred because it becomes difficult to form a particle containing ICG. On the other hand, a construction ratio between the surfactants falling short of the range is not preferred because of the reason that: when the targeting molecule is immobilized, the number of targeting molecules that can be immobilized reduces. As a result, labeling performance for the particle containing ICG reduces.

(Emulsion)

Although the emulsion in the nanoemulsion method may be an emulsion having arbitrary physical properties as long as an object of the present invention can be achieved, the emulsion is preferably an emulsion having a one-peak particle size distribution and having a mean particle size of 1,000 nm or less, more preferably 500 nm or less, still more preferably 200 nm or less.

Such emulsion can be prepared by any one of the conventionally known emulsification approaches such as an intermittent shaking method, a stirring method involving utilizing a mixer such as a propeller type stirring machine or a turbine type stirring machine, a colloid mill method, a homogenizer method, and an ultrasonic irradiation method. One kind of those methods may be employed alone, or two or more kinds thereof may be employed in combination. In addition, the emulsion may be prepared by one-stage emulsification, or may be prepared by multistage emulsification, provided that the emulsification approach is not limited to those approaches as long as the object of the present invention can be achieved.

The emulsion is an oil-in-water (O/W) type emulsion prepared from the mixed liquid obtained by adding the first liquid to the second liquid. Here, the mixing of the first liquid and the second liquid means that the first liquid and the second liquid are caused to exist so as to be in contact with each other without being spatially separated from each other, and does not necessarily require that the liquids be miscible with each other.

Although the ratio between the first liquid and the second liquid in the mixed liquid is not particularly limited as long as the oil-in-water (O/W) type emulsion can be formed, the mixing is preferably performed so that a weight ratio between the first liquid and the second liquid may fall within the range of 1:2 to 1:1,000.

(Solvent Removal)

The solvent removal in the nanoemulsion method is an operation of removing the first liquid from the dispersoid of the emulsion. That is, the solvent removal is to remove the first liquid from the dispersoid constituted of ICG, the additive, and the first liquid (organic solvent).

Although the solvent removal can be performed by any one of the conventionally known methods, a method involving removing the liquid through heating or a method involving utilizing a decompression apparatus such as an evaporator can be given as a method for the solvent removal. Although a heating temperature in the case of the removal through heating is not particularly limited as long as the O/W type emulsion can be maintained, the temperature preferably falls within the range of 0° C. to 80° C., provided that an approach to the solvent removal is not limited to the foregoing as long as the object of the present invention can be achieved.

Although the step of causing ICG to form a J-aggregate is not limited, for example, the warming of the particle and ultrasonic irradiation can each be performed alone or can be performed in combination. Although a temperature condition known in the formation of the J-aggregate of ICG may be used as a warming condition for the particle, the inventors of the present invention have confirmed the formation of the J-aggregate at 37° C. It should be noted that the formation of the J-aggregate of ICG may be performed after the formation of the particle, or may be performed before the formation of the particle.

(Method of Preparing Liposome)

The following two methods are each given as an example of a method of preparing the liposome according to this embodiment: a method involving encapsulating ICG in the liposome and then transforming ICG in the liposome into a J-aggregate, and a method involving encapsulating the J-aggregate of ICG in the liposome.

The liposome can be prepared by a known liposome production method. Examples of the known technology include a method by Bangham (J. Mol. Biol., 13, 238 (1965)) et al., and modifications thereof (Japanese Patent Application Laid-Open No. S52-14013, Japanese Patent Application Laid-Open No. S59-173133, Japanese Patent Application Laid-Open No. H02-139029, and Japanese Patent Application Laid-Open No. H07-241487), an ultrasonic treatment method (Biochem. Biophys. Res. Commun., 94, 1367 (1980)), an ethanol injection method (J. Cell. Biol., 66, 621 (1975)), a cholic acid (surfactant) method (Biochim. Biophys. Acta, 455, 322 (1976)), a freezing and thawing method (Arch. Biochem. Biophys., 212, 186 (1981)), an antiphase evaporation method (Pro. N.A.S. USA, 75, 4194 (1978)), and a method involving using a commercial kit. A liposome prepared by any one of those known methods can be used in this embodiment. That is, ICG can be transformed into a J-aggregate by encapsulating ICG at the time of, or after, the preparation of the liposome and then performing warming or an ultrasonic treatment as required.

A preferred example of the method of producing the liposome encapsulating the J-aggregate of ICG of this embodiment follows the liposome production method reported by Bangham et al. That is, the liposome is formed by: dissolving and mixing raw materials for the liposome such as the phospholipid and a high concentration of ICG in an organic solvent; removing the organic solvent under reduced pressure to dry and harden the lipid and ICG; dispersing the dried and hardened product in an aqueous medium; and uniformizing the resultant through ultrasonic irradiation. After that, the liposome solution can be warmed or subjected to an ultrasonic treatment for transforming ICG into a J-aggregate. The transformation of ICG into the J-aggregate in the liposome may be caused by the aggregation of ICG activated by the warming or the ultrasonic irradiation at the time of the preparation of the liposome or after the preparation.

Otherwise, the following production example of the liposome encapsulating the J-aggregate can be given. Raw materials for the liposome such as the phospholipid are dissolved and mixed in an organic solvent, and then the solutes are dried and hardened by removing the organic solvent under reduced pressure. The dried and hardened product is dispersed in a neutral buffer solution, and then the resultant is uniformized through ultrasonic irradiation to form the liposome. Thus, the liposome containing the neutral buffer solution in itself is prepared. After that, the buffer solution outside the liposome is replaced with an acidic buffer solution. Thus, a liposome dispersion liquid having such a pH gradient that the inside of the liposome is neutral and the outside thereof is acidic is prepared. A solution prepared by dissolving ICG in an acidic buffer solution is added to the liposome dispersion liquid, and then the mixture is warmed and stirred at a temperature equal to or more than the transition temperature of the phospholipid as a raw material for 30 minutes. Thus, ICG that has J-aggregated can be encapsulated in the liposome. A method of encapsulating a drug in a liposome involving utilizing a pH gradient is described in Japanese Patent Translation Publication No. 2006-509769 and is effective for the encapsulation of a basic drug in a liposome. However, ICG is an acidic drug and hence a liposome having a pH gradient opposite to that of the literature was prepared. It was found that when ICG was encapsulated under the condition, most of the encapsulated ICG J-aggregated.

Meanwhile, it is also permitted that ICG is transformed into a J-aggregate in advance and the J-aggregate of ICG is encapsulated in the liposome by the known liposome preparation method. A method of preparing the J-aggregate of ICG is described in Chemical Physics, Volume 220, 1997, Pages 385-392, and for example, a stable J-aggregate of ICG can be prepared by: warming an aqueous solution of ICG (ICG concentration=1.5 mM) in a dark place at 65° C. for 32 hours; and storing the ICG solution at room temperature for 5 days in a dark place. The liposome of this embodiment is obtained by encapsulating the resultant J-aggregate of ICG at the time of the preparation of the known liposome or after the preparation.

The liposome of this embodiment is prepared by purifying the liposome in which the J-aggregate of ICG has been encapsulated as described above through, for example, centrifugal separation, size exclusion chromatography, or ultrafiltration. Important points in the preparation of the liposome encapsulating the J-aggregate of ICG of this embodiment are the following two: an environment having a high concentration of ICG for transforming ICG into a J-aggregate and a stimulus load for the formation of the aggregate through warming or an ultrasonic treatment. As described in Chemical Physics, Volume 220, 1997, Pages 385-392 and Chemical Physics, Volume 220, 1997, Pages 373-384, the concentration of ICG in the solution is at least 0.1 mM or more, preferably 1.0 mM or more, and the warming is performed at not less than 20° C., preferably 37° C. or more, more preferably 65° C. or more. However, a preferred warming temperature ranges from 37° C. to 65° C. because the decomposition of the dye is promoted at 90° C. Although conditions for the ultrasonic treatment are not particularly limited as long as the dye is not decomposed, the treatment is performed under the conditions of, for example, 28 kHz, 30 minutes, and 60° C., or 28 kHz, 60 minutes, and 60° C.

(Contrast Agent)

The particle according to this embodiment can be used as a contrast agent for fluorescent imaging or for photoacoustic imaging because the particle contains the J-aggregate of ICG and can absorb near infrared light to emit fluorescence or an acoustic wave. In addition, the particle can be used as a contrast agent for visual detection because the J-aggregate of ICG is stained dark green.

Here, the “contrast agent” in the description is mainly defined as a substance capable of causing a difference in contrast between a tissue or molecule which one wishes to observe, the tissue or molecule being present in a specimen, and a tissue or molecule around the tissue or molecule to improve the sensitivity of the detection of morphological information or positional information about the tissue or molecule which one wishes to observe. Here, the term “fluorescent imaging” or “photoacoustic imaging” means that the tissue or molecule is imaged with, for example, a fluorescence-detecting apparatus or a photoacoustic signal-detecting apparatus.

A contrast agent according to this embodiment has the particle according to this embodiment and a dispersion medium in which the particle is dispersed. The dispersion medium is a liquid substance for dispersing the particle according to this embodiment, and examples thereof include physiological saline and distilled water for injection. In addition, the contrast agent may have a pharmacologically acceptable additive such as table salt or glucose. The contrast agent according to this embodiment may be such that the particle according to this embodiment is dispersed in the dispersion medium in advance, or may be used as described below. The particle according to this embodiment and the dispersion medium are turned into a kit, and the particle is dispersed in the dispersion medium before administration into a living organism.

(Fluorescence Imaging)

The contrast agent according to this embodiment may also be used for a fluorescence imaging. A fluorescence imaging using the contrast agent according to this embodiment is characterized by including at least the steps of: administering the contrast agent according to this embodiment to a specimen or a sample obtained from the specimen; irradiating the specimen or the sample obtained from the specimen with light; and measuring fluorescence from a substance derived from the particle present in the specimen or in the sample obtained from the specimen.

An example of the fluorescence imaging using the contrast agent according to this embodiment is as described below. That is, the contrast agent according to this embodiment is administered to a specimen, or is added to a sample such as an organ obtained from the specimen. It should be noted that the specimen refers to all living organisms such as a human being, an experimental animal, and a pet without any particular limitation. Examples of the specimen or the sample obtained from the specimen may include an organ, a tissue, a tissue section, a cell, and a cell lysate. After the administration or addition of the particle, the specimen or the like is irradiated with light in a near infrared wavelength region.

In the photoacoustic imaging according to this embodiment, the wavelength of irradiation light may be selected depending on a laser light source to be used. In the fluorescence imaging according to this embodiment, in order to efficiently acquire an acoustic signal, the specimen or the like is preferably irradiated with light having a wavelength of 600 nm to 1,300 nm in a near infrared region called “the optical window” where the influence of absorption and diffusion of light in a living organism is small.

Fluorescence from the contrast agent according to this embodiment is detected and converted to an electrical signal with a fluorescence detector. Based on the electrical signal obtained with the fluorescence detector, the position or size of an absorber in the specimen or the like can be calculated. For example, when the contrast agent is detected above a threshold as a reference, a substance derived from the particle is estimated to be present in the specimen, or a substance derived from the particle can be estimated to be present in the sample obtained from the specimen.

When the contrast agent according to this embodiment is administered to the specimen, a lymph node, in particular, a sentinel lymph node which a cancer cell that has flowed from a cancer primary focus into a lymph duct reaches first can be suitably detected. In this case, a contrast agent for a lymph node is injected into a tumor or around the tumor, and the detection of the contrast agent is performed at an appropriate time after the injection.

(Photoacoustic Imaging)

The contrast agent according to this embodiment may be used for a photoacoustic imaging. It should be noted that the term “photoacoustic imaging” as used herein is a concept including photoacoustic tomography (tomogram method). A photoacoustic imaging using the contrast agent according to this embodiment is characterized by including at least the steps of: administering the contrast agent according to this embodiment to a specimen or a sample obtained from the specimen; irradiating the specimen or the sample obtained from the specimen with pulse light; and measuring a photoacoustic signal from a substance derived from the particle present in the specimen or in the sample obtained from the specimen.

An example of the photoacoustic imaging using the contrast agent according to this embodiment is as described below. That is, the contrast agent according to this embodiment is administered to a specimen, or is added to a sample such as an organ obtained from the specimen. It should be noted that the specimen refers to all living organisms such as a human being, an experimental animal, and a pet without any particular limitation. Examples of the specimen or the sample obtained from the specimen may include an organ, a tissue, a tissue section, a cell, and a cell lysate. After the administration or addition of the particle, the specimen or the like is irradiated with laser pulse light having a wavelength in a near infrared region.

In the photoacoustic imaging according to this embodiment, the wavelength of irradiation light may be selected depending on a laser light source to be used. In the photoacoustic imaging according to this embodiment, in order to efficiently acquire an acoustic signal, the specimen or the like is preferably irradiated with light having a wavelength of 600 nm to 1,300 nm in a near infrared region called “the optical window” where the influence of absorption and diffusion of light in a living organism is small.

A photoacoustic signal (acoustic wave) from the contrast agent according to this embodiment is detected and converted to an electrical signal with an acoustic wave detector such as a piezoelectric transducer. Based on the electrical signal obtained with the acoustic wave detector, the position or size of an absorber in the specimen or the like, or the optical characteristic value distribution of a molar absorption coefficient or the like can be calculated. For example, when the contrast agent is detected above a threshold as a reference, a substance derived from the particle is estimated to be present in the specimen, or a substance derived from the particle can be estimated to be present in the sample obtained from the specimen.

When the contrast agent according to this embodiment is administered to the specimen, a lymph node, in particular, a sentinel lymph node which a cancer cell that has flowed from a cancer primary focus into a lymph duct reaches first can be suitably detected. In this case, a contrast agent for a lymph node is injected into a tumor or around the tumor, and the detection of the contrast agent is performed at an appropriate time after the injection.

In this embodiment, quenching due to the accumulation of the dye is caused by suppressing the leakage of the dye, and hence the energy of the applied pulse light is prevented from being used in fluorescent emission and can be converted into an additionally large quantity of thermal energy. Consequently, an acoustic signal can be acquired in an additionally efficient manner.

### Embodiment 2

Another example of the particle according to an embodiment of the present invention is based on the new finding that the retention ratio of ICG of an ICG-encapsulating particle whose ratio of its absorbance for light having a wavelength of 700 nm (derived from an H-aggregate of ICG) to its absorbance for light having a wavelength of 780 nm (derived from a monomer of ICG) (also referred to as “700/780 ratio” in this embodiment) shows a high value of 1 or more is kept high even in a serum.

Therefore, the preparation of the ICG-encapsulating particle having a 700/780 ratio of 1 or more can provide such a contrast agent using the ICG-encapsulating particle that the leakage of ICG in a serum is suppressed.

It has been known that although the monomer of ICG has a maximum absorption wavelength of about 780 nm, the formation of an H-aggregate causes the wavelength to shift to shorter wavelengths, specifically, about 700 nm. Therefore, when the 700/780 ratio of the particle is high, ICG to be encapsulated in a lipid particle is assumed to form an H-aggregate.

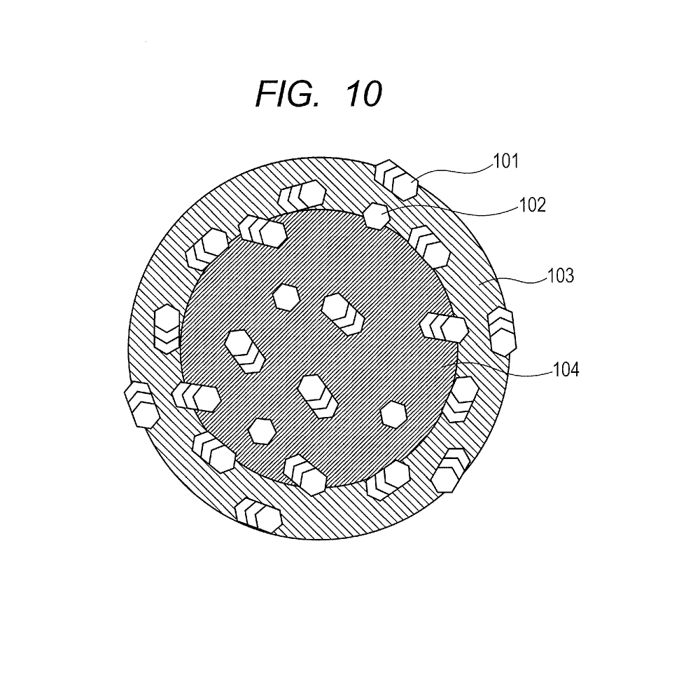

FIG. 10 illustrates an example of the particle of the present invention based on a liposome. An ICG 101 as an H-aggregate is encapsulated in a liposome covered with a membrane 103. The membrane 103, which is a bilayer membrane based on a phospholipid here, may be a membrane formed of any other component. The ICG 101 as an H-aggregate, which is encapsulated in an internal aqueous phase 104 of the liposome, may be present in the membrane 103 or on its surface. In addition, not all ICG's are needed to be H-aggregates, and an ICG as a form except an H-aggregate, e.g., an ICG 102 as a monomer may be present as long as its 700/780 ratio is 1 or more.

(Particle)

The particle in this embodiment is a particle containing at least a phospholipid and cholesterol, and such a lipid vesicle or liposome that a lipid is a main constituent of a membrane is also included in the category thereof. Although the liposome typically means a lipid vesicle in which a bilayer membrane constituted mainly of a phospholipid is constituted of one or more membranes, the term “particle” as used in this embodiment comprehends all kinds of particles each containing at least a phospholipid and cholesterol. According to the findings of the inventors of the present invention, in the particle of this embodiment, cholesterol may contribute to the formation of an H-aggregate and the stabilization of the particle to increase the 700/780 ratio. It should be noted that even when ICG enters a membrane of a particle to disturb the order of the membrane, the particle is included in the category of the particle as used in this embodiment as long as the particle is dispersed in a dispersion medium so that its particle size may fall within the following range. In addition, the particle may contain a lipid, a glycolipid, a sterol derivative, a lipid derivative, or a combination thereof as a constituent. The particle is preferably a lipid particle using a lipid as a main constituent. At this time, a particle membrane and the other constituent may be constituted of a mixture of different lipids. In addition, for example, a polyethylene glycol-bonded phospholipid can be used as a lipid derivative.

The particle may contain a surfactant on its surface, and a target site can be specifically labeled by immobilizing a targeting molecule to part of the particle.

The particle may be prepared by a conventionally known method, and a method for the preparation can be appropriately selected in order that a particle having desired physical properties may be obtained. The kind, amount, and the like of a constituent such as a lipid can be appropriately selected according to the applications of the particle. In the case of, for example, a lipid particle, the particle size (hereinafter, the mean value of particle diameters is referred to as “particle size”) and surface potential of the particle can be controlled by taking the kind of a lipid, the amount of the lipid, the ratio thereof, and the charge of the lipid into consideration.

Preferred examples of the phospholipid in the particle of this embodiment include synthetic distearoylphosphatidylcholine (DSPC), and there may be also used any other alkyl and alkenyl derivatives of synthetic phosphatidic acid (PA) or the like. Examples thereof include dimyristoylphosphatidylcholine (DMPC), dipalmitoylphosphatidylcholine (DPPC), dioleylphosphatidylcholine (DOPC), distearoylphosphatidylserine (DSPS), distearoylphosphatidylglycerol (DSPG), and dipalmitoylphosphatidic acid (DPPA).

Examples of the other phospholipid may include: a soy or yolk lecithin, a lysolecithin, and a derivative of a hydrogenated product or hydroxide thereof; and a semisynthetic phosphatidylcholine, a phosphatidylserine (PS), a phosphatidylethanolamine, a phosphatidylglycerol (PG), a phosphatidylinositol (PI), and a sphingomyelin.

Examples of the glycolipid to be included in the constituent include: a glycerolipid such as digalactosyldiglyceride; and sphingoglycolipids such as a galactosylceramide and a ganglioside.

As a constituent material for the particle, any other material may be added as required. Examples thereof include: a glycol such as ethylene glycol acting as a membrane stabilizing agent; a phosphoric acid dialkyl ester added to control a charge; and an aliphatic amine such as stearylamine.

(Encapsulation of ICG in Particle)

ICG to be encapsulated in the particle is a water-soluble substance and is hence typically encapsulated in an internal aqueous phase in the case of, for example, a liposome-like lipid particle. In addition, ICG can be encapsulated in the particle by filling the inside of the particle with a polymer or the like. However, ICG has an affinity for a phospholipid, and hence its presence on the surface of a lipid membrane or in a lipid bilayer membrane can occur. In this embodiment, the three cases, i.e., “encapsulation in the particle,” “presence in the membrane of the particle,” and “presence on the surface of the particle” are collectively referred to as “encapsulation.”

The particle according to this embodiment is a particle encapsulating ICG and the ratio at which the ICG forms an H-aggregate is assumed to be high.

In this embodiment, the H-aggregate of ICG refers to a multimer of ICG but a monomer of ICG may be included. That is, the abundance ratio of the monomer is not particularly limited as long as the ICG-encapsulating lipid particle according to this embodiment has a ratio of its absorbance for light having a wavelength of 700 nm (derived from the H-aggregate) to its absorbance for light having a wavelength of 780 nm (derived from the monomer) of 1 or more.

(Preparation Method of ICG-Encapsulating Particle Showing 700/780 Ratio of 1 or More)

The preparation method for the particle according to this embodiment is not particularly limited, and the particle can be prepared by, for example, a known liposome production method. Examples thereof include Bangham's methods (a simple hydration method, a sonication method, and an extrusion method), a pH gradient (remote loading) method, a counter ion concentration gradient method, a freeze-thaw method, an antiphase evaporation method, a mechanochemical method, a supercritical carbon dioxide method, and a film loading method, and a method using a commercially available hollow liposome. The particle prepared by any of these known methods can be used in this embodiment.

It has been found that ICG forms an H-aggregate when its concentration in a solution increases. In this embodiment, however, it is assumed that a 700/780 ratio of 1 or more leading to the formation of the H-aggregate can be achieved with cholesterol and a dextran to be arbitrarily added without any increase of the ICG concentration in the solution.

A preferred example of the method of producing the ICG-encapsulating particle showing a 700/780 ratio of 1 or more of this embodiment follows the liposome production method based on Bangham's method. That is, the liposome is formed by: dissolving and mixing raw materials for the particle such as the phospholipid and a high concentration of ICG in an organic solvent; removing the organic solvent under reduced pressure to dry and harden the raw materials for the particle; dispersing the dried and hardened product in an aqueous medium; and uniformizing the resultant through ultrasonic irradiation. At this time, the 700/780 ratio can be increased by adding the dextran to the aqueous medium.

A preparation method in conformity with an antiphase evaporation method is also given as another preferred example of the method of producing the ICG-encapsulating particle showing a 700/780 ratio of 1 or more of this embodiment. That is, raw materials for the particle such as the phospholipid and a high concentration of ICG are dissolved in an organic solvent that has difficulty in freely mixing with water (such as chloroform), the solution is dropped in an aqueous medium, and the mixture is irradiated with an ultrasonic wave to prepare an O/W emulsion. After that, the organic solvent is removed under reduced pressure and then the remainder is subjected to a purifying step described in examples to be described later. Thus, the ICG-encapsulating lipid particle can be prepared. Although a W/O/W emulsion is typically used in the preparation of a liposome by the antiphase evaporation method, it has been found that the ICG-encapsulating particle can be produced even with the O/W emulsion in this embodiment. The method enabled the preparation of an ICG-encapsulating particle having a 700/780 ratio of 1 or more without the addition of any dextran to the aqueous medium. However, the 700/780 ratio was able to be additionally increased by adding the dextran to the aqueous medium.

It should be noted that the organic solvent that has difficulty in freely mixing with water is an organic solvent that is capable of dissolving the mixture of ICG and the raw materials for the particle such as the phospholipid and cholesterol, and that has no solubility or small solubility in water. Specific examples of such organic solvent include halogenated hydrocarbons (such as dichloromethane, chloroform, chloroethane, dichloroethane, trichloroethane, and carbon tetrachloride). One kind of such hydrophobic solvents may be used, or two or more kinds thereof may be used after having been mixed at an appropriate ratio, provided that the solvent is not limited to the specific examples.

(Particle Size)

The particle size of the particle according to this embodiment is not particularly limited, provided that when the particle is used as a contrast agent, in particular, a contrast agent for a lymph node, setting its hydrodynamic mean particle size to 1,000 nm or less can enhance the ease with which the particle is taken in a lymph duct or a tissue (tissue permeability) and its retentivity in a lymph node or the tissue.

When the particle size is 1,000 nm or less, a larger amount of particles can be accumulated in a tumor site than that in a normal site in a living organism by an enhanced permeability and retention (EPR) effect. The tumor site can be specifically imaged by detecting the accumulated particles with various image-forming modalities such as fluorescence and photoacoustics. In addition, when the particle size exceeds 1,000 nm, efficient intake in a tissue such as a lymph duct cannot be expected. Consequently, the mean particle size is preferably set to 10 nm or more and 1,000 nm or less. The mean particle size is more preferably 20 nm or more and 500 nm or less, still more preferably 20 nm or more and 200 nm or less, particularly preferably 20 to 100 nm. This is because when the particle size of the particle is 200 nm or less, the particle is hardly taken in a macrophage in blood and hence its retentivity in the blood may improve.

The particle size can be measured through observation with an electron microscope or by a particle size-measuring method based on a dynamic light scattering method. When the particle size is measured based on the dynamic light scattering method, a hydrodynamic diameter is measured with a dynamic light scattering analyzer (DLS-8000, manufactured by Otsuka Electronics Co., Ltd.) by the dynamic light scattering (DLS) method.

(Dextran)

The term “dextran” as used herein refers to a compound represented by the following chemical formula 7.

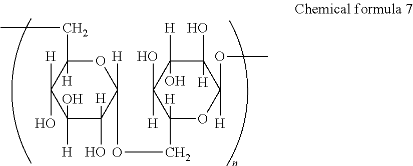

The nomenclature of the dextran changes between dextran 40 (having a molecular weight of 40 kDa) and dextran 70 (having a molecular weight of 70 kDa) depending on a difference in n, i.e., a difference in molecular weight.

The particle according to this embodiment is preferably such that the dextran has been dissolved in the aqueous medium to be used at the time of the preparation of the particle. Although any dextran can be used as long as the dextran can be dissolved in the aqueous medium, a preferred molecular weight is 20 kDa to 100 kDa, and dextran 40 (having a molecular weight of 40 kDa) and dextran 70 (having a molecular weight of 70 kDa) described in the Japanese Pharmacopoeia can each be given as an optimum dextran.

The concentration of the dextran to be used at the time of the preparation of the particle is 1.5 wt % or more with respect to the aqueous medium and an effective concentration is 2.6 wt % or more. The case where the highest effect is observed is as described below. The addition of the dextran at a concentration of 13 wt % can increase the 700/780 ratio.

(Retention Ratio of ICG)

In this embodiment, the difficulty with which ICG leaks from the particle can be determined by a retention ratio of ICG after the particle has been incubated in a serum solution at 37° C. for 24 hours. The retention ratio of ICG represents the ratio of ICG remaining in the particle without leaking, and as described in the examples to be described later, the ratio of ICG that leaks from the particle into the solvent after the incubation for 24 hours is determined from the integrated value of the light absorption spectrum of the solution in the wavelength range of 650 to 900 nm and the integrated value of the light absorption spectrum of a supernatant after the precipitation of the particle by centrifugal separation in the wavelength range of 650 to 900 nm.

The particle of this embodiment has a retention ratio of ICG of 60% or more, preferably 70% or more, more preferably 90% or more.

(Polyethylene Glycol)

A polyethylene glycol is preferably introduced into the surface of the membrane of the particle according to this embodiment. An example of the applications of the particle of this embodiment is a tumor contrast agent. In order that the enhanced permeability and retention (EPR) effect proposed as a principle of passive targeting to a tumor may be caused, the contrast agent is required to have high retentivity in blood. The introduction of the polyethylene glycol into the lipid particle of this embodiment is extremely useful because of the reason that: when its interaction with a protein in blood such as a complement is suppressed, the polyethylene glycol is hardly phagocytosed by a reticuloendothelial cell of a liver or the like and hence can improve the retentivity of the particle in the blood.

A function of the polyethylene glycol can be regulated by appropriately changing its molecular weight and its ratio of introduction into the particle. A polyethylene glycol having a molecular weight of 500 to 200,000 is preferably used and the molecular weight is particularly suitably 2,000 to 100,000. In addition, when the polyethylene glycol is introduced into a lipid particle, the ratio of introduction thereof is preferably 0.001 to 50 mol %, more preferably 0.01 to 30 mol %, still more preferably 0.1 to 10 mol % with respect to a lipid constituting the lipid particle.

Any known technique can be used as a method of introducing a polyethylene glycol into the particle. Preferred examples thereof include a method involving incorporating a polyethylene glycol-bonded phospholipid or the like into the phospholipid as the particle raw material in advance to produce the particle. Examples of the polyethylene glycol-bonded phospholipid include a polyethylene glycol derivative of a phosphatidylethanolamine, such as a distearoylphosphatidylethanolamine-polyethylene glycol (DSPE-PEG).

(Particle Size Reduction Treatment)

Meanwhile, in order that the enhanced permeability and retention (EPR) effect proposed as a principle of passive targeting to a tumor may be caused, it is advantageous that the contrast agent has a small particle size because tumor vascular permeability is enhanced. It has been known that, for example, when a particle (such as a liposome) encapsulating ICG is transferred to a tissue in a living organism which one wishes to contrast by means of intravenous injection, a particle size for increasing the amount of particles to be transferred to the tissue in the living organism which one wishes to contrast has an optimum value. When the particle size is excessively large, the particle hardly exits a vessel and is metabolized by the phagocytosis of a Kupffer cell during its retention in the vessel, and hence the transfer amount reduces. In contrast, when the particle size is excessively small, the concentration of the particle in blood reduces owing to excretion in a kidney and hence the transfer amount reduces as well.

The size of a liposome may be determined depending on a spontaneous curvature based on the morphology of a lipid molecule containing, for example, hydration water in an equilibrium state in principle. That is, the liposome is such that an amphipathic molecule forms a vesicle in a self-organizing manner, and hence its particle size is included in a range having a distribution with a mean value, which depends on an amphipathic material, solvent composition, and an environmental factor such as a concentration, a temperature, or a pressure, at its center.

The size of the liposome is strongly affected by a process leading to the formation (such as a preparation operation) as well. In ordinary cases, a giant unilamellar vesicle (GUV) is formed by a gentle hydration method under a mild condition, a multilamellar vesicle (MLV) is formed under a vortex flow like a vortex treatment, and a small unilamellar vesicle (SUV) is formed by a high-output ultrasonic treatment.

After the formation of the vesicle, an instrument called an extruder has been generally used in a particle size reduction treatment for intentionally reducing a (mean) particle size or a sizing treatment for uniformizing a particle size distribution so that the distribution may be small (narrow). The extruder is an instrument for reducing the particle size of, and sizing, a particle by: warming the liposome to a temperature equal to or more than its phase transition temperature to bring the liposome into a state where the liposome can be easily plasticized; and then passing the liposome through a pore having a specific size. For example, treating an ICG-encapsulating particle prepared by the preparation method with an extruder having a mean pore size of 30 nm provides a particle having a particle size of 120 to 150 nm.

Meanwhile, in this embodiment, a liposome particle having a particle size in an optimum range additionally effective for a contrast agent can be provided by: diluting ICG-encapsulating particles prepared by the preparation method with an aqueous buffer solution; removing particles each having a size of 1 μm or more through filtration with a filter having a pore size of 1 μm or less; and redispersing the remainder with an ultrasonic wave. According to the particle size reduction treatment in this embodiment, particles each having a particle size additionally suitable for a contrast agent, the particles having a mean particle size of 50 to 60 nm, can be obtained. The term “mean particle size” as used herein refers to the mean particle size of a particle size distribution based on cumulant analysis measured by the dynamic light scattering method (DLS method).

The ratio of the dilution with the aqueous buffer solution is particularly suitably about 10-fold, and suitably falls within the range of 4-fold to 100-fold. The effect of the dilution lies in the suppression of the agglomeration of particles, and their agglomeration property is not uniform because the agglomeration property depends on a material constituting the liposome, the initial concentration and initial particle size of the liposome, and an environmental factor. As a comparative experiment, the particles were passed through the filter alone without being diluted, and were then subjected to the ultrasonic redispersion. As a result, their particle sizes did not reduce. That is, the initial particle size showed no change even after the redispersion because the size adopted an optimum value in the environment. In the particle size reduction treatment of this embodiment, the optimum particle size may shift to smaller values by virtue of an environmental change called the dilution (change in number of liposomes in a volume).

A neutral aqueous buffer solution such as 2-[4-(2-hydroxyethyl)-1-piperazinyl]-ethanesulfonic acid (HEPES) is preferred as the aqueous buffer solution in consideration of an influence at the time of administration to a living organism.

Subsequently, the particles each having a size of 1 μm or more are removed through the filtration with the filter having a pore having a pore size of 1 μm or less. The pore size of the filter is suitably 0.2 μm or more and 1 μm or less. The step may have a reducing effect on the initial particle size before the redispersion with an ultrasonic apparatus. Although a dispersion treatment with the ultrasonic apparatus is credited with having a shearing action on the liposome, it can be said that the amount of a small component or fraction increases as the initial particle size reduces.

A bath type ultrasonic apparatus is suitably used in the redispersion and the treatment is preferably performed at a frequency in the range of 20 kHz to 100 kHz. Switching among multiple frequencies in the range is particularly preferably performed every fixed time. An apparatus having an output of 100 W to 200 W is generally available as long as the apparatus is manufactured in Japan, though the output is not particularly limited.

The particle in this embodiment is a particle containing at least a phospholipid and cholesterol, and such a lipid vesicle or liposome that a lipid is a main constituent of a membrane is also included in the category thereof. Although the liposome typically means a lipid vesicle in which a bilayer membrane constituted mainly of a phospholipid is constituted of one or more membranes, the term “particle” as used in this embodiment comprehends all kinds of particles each containing at least a phospholipid and cholesterol. Consequently, a treatment temperature is not limited to the phase transition temperature of the liposome and a temperature equal to or more than the temperature is permitted. For example, when the phospholipid is distearoyl phosphatidylcholine (DSPC), the treatment temperature is 40° C. to 70° C., particularly preferably 55° C. or more and 65° C. or less.

The bath type ultrasonic apparatus is suitably used in the redispersion because the liposome dispersion liquid whose volume has been increased by the dilution can be uniformly subjected to an ultrasonic dispersion treatment. Although an ultrasonic dispersing apparatus of a type called a probe type has also been known, the apparatus is generally unfit for large-volume dispersion because of the reason that the intensity of an ultrasonic wave attenuates in proportion to the square of a distance and hence a treatment effect is limited within a distance range near a probe in the probe type apparatus. The bath type apparatus can spread an ultrasonic wave over nearly the entire surface of an ultrasonic bath.

An apparatus capable of using ultrasonic waves having multiple frequencies in combination is suitably used for the large-volume dispersion because a wide range can be treated with a low-frequency ultrasonic wave and a local range can be intensively treated with a high-frequency ultrasonic wave.

In the ultrasonic dispersion treatment of a liposome, the temperature of a liquid in a bath is increased so as to be equal to or more than the phase transition temperature of a substance constituting the liposome in many cases. In contrast, in the case of the bath type apparatus, temperature control at the time of the treatment is easy, and the temperature of a liquid in a bath can be easily kept constant irrespective of a place in the bath and a time, which is convenient.

In the particle size reduction treatment, a particle having a small particle size can be obtained probably because the initial particle size before the redispersion is reduced by: suppressing the agglomeration of particles through the dilution with the aqueous buffer solution; and removing particles each having a size of 1 μm or more through the filtration with the filter. The particle size reduction treatment is a method suitable for the case where ICG is included in a particle or the case where a phospholipid, cholesterol, a DSPE-PEG, or the like is used as a surfactant. In addition, the particle size reduction treatment is a method suitable upon reduction in particle size of a particle prepared by Bangham's method or the nanoemulsion method.

A particle whose particle size has been reduced as described above can be used in a contrast agent having high tissue permeability.

(Contrast Agent)

The particle according to this embodiment can be used as a contrast agent for fluorescent imaging or for photoacoustic imaging because the particle encapsulates ICG and hence can absorb near infrared light to emit fluorescence or an acoustic wave. In addition, the particle according to this embodiment can be used as a contrast agent for visual detection because the particle is stained dark green.

Here, the “contrast agent” in the description is mainly defined as a substance capable of causing a difference in contrast between a tissue or molecule which one wishes to observe, the tissue or molecule being present in a specimen, and a tissue or molecule around the tissue or molecule to improve the sensitivity of the detection of morphological information or positional information about the tissue or molecule which one wishes to observe. Here, the term “fluorescent imaging” or “photoacoustic imaging” means that the tissue or molecule is imaged with, for example, a fluorescence-detecting apparatus or a photoacoustic signal-detecting apparatus.

A contrast agent according to this embodiment may have the particle according to this embodiment and a dispersion medium in which the particle is dispersed. The dispersion medium is a liquid substance for dispersing the particle according to this embodiment, and examples thereof include physiological saline and distilled water for injection. The contrast agent according to this embodiment may be such that the particle according to this embodiment is dispersed in the dispersion medium in advance, or may be used as described below. The particle according to this embodiment and the dispersion medium are turned into a kit, and the particle is dispersed in the dispersion medium before administration into a living organism.

The contrast agent using the particle according to this embodiment as a main component may have a pharmacologically acceptable additive. Examples thereof include: isotonizing agents such as saccharides including sucrose and glucose, and polyhydric alcohols including glycerin and propylene glycol; pH adjustors; and stabilizers. The contrast agent and any additive can be used as a mixture before the administration into the living organism.

An imaging method involving using the contrast agent using the particle according to this embodiment as a main component includes the steps of: administering the contrast agent to a subject; accumulating the contrast agent in a target tissue; and detecting the contrast agent present in the target tissue. A method of detecting the contrast agent is, for example, a direct observation method with the naked eye, a near infrared fluorescence method, or a photoacoustic method.

An example of a photoacoustic imaging according to this embodiment is as described below. That is, the contrast agent having the particle according to this embodiment is administered to a specimen. It should be noted that examples of the specimen include, but not particularly limited to, mammals such as a human being, an experimental animal except the human being, and a pet, and other animals. The administration may be in vivo administration, or may be in vitro administration. After the administration of the contrast agent, the specimen or the like is irradiated with laser pulse light in a near infrared wavelength region. Next, a photoacoustic signal (acoustic wave) from the contrast agent is detected with an acoustic wave detector such as a piezoelectric transducer to be converted into an electric signal. The position or size of an absorber in the specimen or the like, or the optical characteristic value distribution of an absorption coefficient or the like can be calculated based on the electric signal obtained with the acoustic wave detector. An example of the preferred applications of the contrast agent using the lipid particle according to this embodiment as a main component is to detect a tumor.

It should be noted that an example of a fluorescence imaging according to this embodiment is as described below. In the photoacoustic imaging, the specimen or the like is irradiated with excitation light instead of the irradiation with laser pulse light in a near infrared wavelength region, and then fluorescence from the contrast agent is detected.

### Examples Corresponding to Embodiment 1

Specific reagents and reaction conditions to be used upon production of particles each containing a J-aggregate of ICG are given in the following examples. However, these reagents and reaction conditions can be modified, and such modifications are included in the scope of the present invention. Therefore, the following examples are intended to aid the understanding of the present invention and by no means limit the scope of the present invention.

### Example 1

### Example 1-1

### Preparation of J-Aggregate of ICG

The preparation of a J-aggregate of ICG was performed with reference to the method described in Chemical Physics, Volume 220, 1997, Pages 385-392. First, 20 mL of distilled water were added to 23.4 mg of ICG and then the mixture was irradiated with an ultrasonic wave for 3 minutes to prepare a 1.5-mM aqueous solution of ICG. The aqueous solution of ICG was warmed in a dark place at 65° C. for 24 hours. Next, the aqueous solution of ICG was left at rest in a dark place at room temperature for 5 days to provide a J-aggregate of ICG. Hereinafter, the J-aggregate of ICG thus prepared is abbreviated as “J-ICG.” An aqueous solution of the J-ICG was kept in cold storage after the preparation.

(Filtration with Pore Filter)

In order for a particle containing a J-aggregate of ICG according to the present invention to be obtained, the aqueous solution of the J-ICG was filtered with a pore filter and then the filtrate was recovered. The pore sizes of the pore filters used here were 1.2 μm, 0.45 μm, 0.2 μm, and 0.1 μm, and respective J-ICG's obtained through the filtration with the pore filters are hereinafter abbreviated as “J-ICG-1.2 μm,” “J-ICG-0.45 μm,” “J-ICG-0.2 μm,” and “J-ICG-0.1 μm.”

(Absorption Spectrum Measurement)

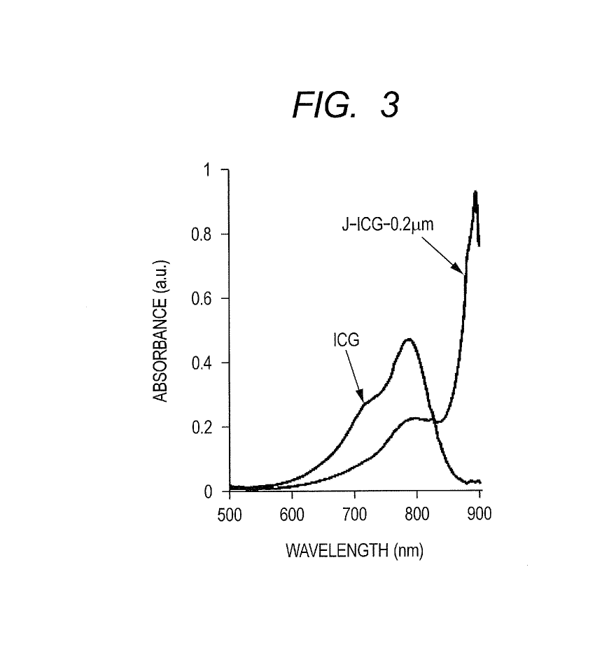

The absorbances at 780 nm and 895 nm of the resultant J-ICG, J-ICG-1.2 μm, J-ICG-0.45 μm, J-ICG-0.2 μm, and J-ICG-0.1 μm were measured. Hereinafter, in the description, a GeneQuant 100 (manufactured by GE Healthcare Japan) was used in the measurement of an absorption spectrum and an absorbance unless otherwise stated. As a reference, the aqueous solution of ICG was similarly subjected to the measurement. FIG. 3 shows examples of the absorption spectra of the J-ICG-0.2 μm and the aqueous solution of ICG. Absorption at 780 nm is derived from a monomer of ICG (sometimes referred to as “monomer”) and that at 895 nm is derived from the J-aggregate. That is, a ratio of the absorbance at 895 nm to the absorbance at 780 nm may represent the formation ratio of the J-aggregate. Table 1 shows the absorbance at 895 nm (sometimes abbreviated as “Abs895”) and absorbance at 780 nm (sometimes abbreviated as “Abs780”) of each aqueous solution, and a ratio between the absorbances (sometimes abbreviated as “Abs895/Abs780”). It should be noted that in the measurement, a quartz cell having an optical path length of 1 cm was used, and an aqueous solution prepared by diluting the aqueous solution of the J-ICG with distilled water about 400-fold and an aqueous solution prepared by diluting the J-ICG-0.1 μm with distilled water 200-fold were used. As is apparent from Table 1, in the J-ICG, the absorption by the monomer reduced as compared with ICG and the absorption at Abs895 nm appeared, and hence the presence of the J-aggregate was able to be confirmed. In addition, the Abs895/Abs780 showed a value of 5.0 to 6.0. In the aqueous solution of ICG, the absorption at Abs895 nm did not appear because no J-aggregate was formed.

TABLE 1

| | Abs895 | Abs780 | Abs895/Abs780 |
|---|---|---|---|
| J-ICG | 0.75 | 0.13 | 5.8 |
| J-ICG-1.2 μm | 0.73 | 0.14 | 5.2 |
| J-ICG-0.45 μm | 0.68 | 0.14 | 4.9 |
| J-ICG-0.2 μm | 0.82 | 0.18 | 4.6 |
| J-ICG-0.1 μm | 1.04 | 0.17 | 6.1 |
| ICG | 0.0 | 0.3 | 0 |

(Particle Size Measurement)

The particle sizes of the resultant J-ICG, J-ICG-1.2 μm, J-ICG-0.45 μm, J-ICG-0.2 μm, and J-ICG-0.1 μm were measured by the DLS method. As a reference, the aqueous solution of ICG was similarly subjected to the measurement. As is apparent from Table 2, particle formation (aggregate formation) was not observed in ICG, but in the J-ICG, a particle size of about 3 microns was calculated and an extremely polydisperse particle size distribution was obtained. According to a prior literature, the J-ICG was reported to be a structural body of several microns, which coincided with the result. On the other hand, the J-ICG-1.2 μm, J-ICG-0.45 μm, J-ICG-0.2 μm, and J-ICG-0.1 μm filtered with the pore filters showed particle sizes dependent on the pore sizes of the filters. The J-ICG-0.1 μm had a particle size of 293 nm and its polydispersity index unexpectedly reduced to 0.2, and hence it was found that a particle size distribution was improved as compared with that before the filtration with the pore filter. In addition, the particle sizes of the J-ICG-1.2 μm, the J-ICG-0.45 μm, and the J-ICG-0.2 μm were observed for 7 days. As a result, the particle sizes showed no changes and were stable.

TABLE 2

| | Cumulant particle | Polydispersity index |
|---|---|---|
| | size (nm) | (PDI) |
| J-ICG | 3100 | 0.81 |
| J-ICG-1.2 μm | 1077 | 0.38 |
| J-ICG-0.45 μm | 680 | 0.30 |
| J-ICG-0.2 μm | 312 | 0.19 |
| J-ICG-0.1 μm | 293 | 0.22 |
| ICG | Undetectable | Undetectable |

(Evaluation for Stability of Aggregate in Serum)

The J-ICG, and the J-ICG-0.2 μm and J-ICG-0.1 μm according to the present invention obtained in the foregoing were each evaluated for the stability of the aggregate in a serum. As a comparative example, ICG was similarly subjected to the measurement. An evaluation method is as described below. First, 0.1 mL of each sample aqueous solution was transferred to a sample tube and then 0.9 mL of a bovine serum was added to the solution. Next, the mixture was incubated in a dark plate at 37° C. for 24 hours. Next, the absorbance at 895 nm (sometimes abbreviated as “Abs895”) and absorbance at 800 nm (sometimes abbreviated as “Abs800”) of each aqueous solution were measured. Next, the sample was subjected to ultracentrifugation (280,000 G, 17 minutes, room temperature, sample volume: 0.5 mL) to precipitate the J-aggregate. After the centrifugation, the absorbance of the supernatant was measured. A himac CS150GXL (manufactured by Hitachi Koki Co., Ltd.) was used as a centrifugal separator. Under the centrifugation conditions, most of ICG precipitates, and on the other hand, most of the J-aggregate precipitates. Therefore, if the J-aggregate collapses in the serum to return to an ICG monomer, the precipitation amount reduces, and the absorbances of ICG of the solution (in the post-centrifugation case, the supernatant) before and after the centrifugation become equal to those in the case of ICG. Therefore, the stability of the aggregate is defined as described below. When the aggregate is stable, the stability is 1, and when the aggregate is unstable, the stability reduces to 0. Stability of aggregate (in serum)=1−(absorbance at 800 nm of centrifugation supernatant after centrifugation/absorbance at 800 nm before centrifugation)

TABLE 3

| | J-ICG | J-ICG-0.2 μm | J-ICG-0.1 μm | ICG |
|---|---|---|---|---|
| Stability of | 0.79 | 0.86 | 0.87 | 0.19 |
| aggregate | | | | |

As is apparent from Table 3, the J-ICG is stable even in the serum and dissociates into the ICG monomer to an extremely small extent. The stability of each of the J-ICG-0.2 μm and J-ICG-0.1 μm according to the present invention was found to be higher than that of the J-ICG. An aggregate having a smaller size may have higher stability in the serum and hence the result may reflect that the ratio of a small aggregate was increased by the filtration with the pore filter.

(Evaluation for Accumulation Ratio in Lymph Node)

Each of the resultant J-ICG-1.2 μm and J-ICG-0.2 μm was subcutaneously administered to a planta of a mouse, and 24 hours after the administration, its accumulation ratio in a lymph node below the knee was measured. The administration amount was set to 13 nmol in terms of ICG. As a comparative example, the aqueous solution of ICG was similarly subjected to the measurement. As is apparent from Table 4, the accumulation ratio of each of the J-ICG-1.2 μm and J-ICG-0.2 μm in the lymph node increased as compared with that of ICG. In addition, the dependence of the accumulation ratio on the size of the J-ICG was observed and a J-ICG having a smaller size showed a better accumulation ratio in the lymph node. The foregoing results have shown that the particle of the present invention in which the size of the J-aggregate of ICG is set to several hundreds of nanometers can function as a contrast agent for a lymph node.

TABLE 4

| | Accumulation ratio in lymph node (%) |
|---|---|
| J-ICG-1.2 μm | 0.14 |
| J-ICG-0.2 μm | 0.31 |
| ICG | 0.10 |

(Fluorescent Imaging of Lymph Node)

Each of the J-ICG-1.2 μm and the J-ICG-0.2 μm was subcutaneously administered to a planta of a mouse, and 24 hours after the administration, a lymph node below the knee was extirpated and subjected to fluorescent imaging. As a result, a fluorescence signal from the lymph node was confirmed. A fluorescence signal from a non-administered lymph node as a comparative example was not confirmed. The foregoing results have shown that the particle of the present invention can function as a fluorescent contrast agent for lymph node imaging.

(Measurement of Photoacoustic Signal)

The intensity of the photoacoustic signal of the J-ICG-0.2 μm was measured. As a comparative example, the aqueous solution of ICG was similarly subjected to the measurement. The measurement of the photoacoustic signal was performed by: irradiating the sample with pulse laser light; detecting the photoacoustic signal from the sample with a piezoelectric element; amplifying the signal with a high speed preamplifier; and acquiring the amplified signal with a digital oscilloscope. Specific conditions for the measurement are as described below. Titanium sapphire laser (manufactured by Lotis Ltd.) was used as a light source. The conditions of a wavelength of 780 nm or 895 nm, an energy density of 12 mJ/cm², a pulse width of 20 nanoseconds, and a pulse repetition of 10 Hz were adopted. A Model V303 (manufactured by Panametrics-NDT) was used as an ultrasonic transducer. The conditions of a central band of 1 MHz, an element size of φ0.5, a measurement distance of 25 mm (non-focus), and an amplification of +30 dB (Ultrasonic Preamplifier Model 5682 manufactured by Olympus Corporation) were adopted. A measurement vessel was a cuvette made of a polystyrene, and had an optical path length of 0.1 cm and a sample volume of about 200 μl . Water was used as a solvent. A DPO4104 (manufactured by TEKTRONIX, INC.) was used as a measuring device, and measurement was performed under the conditions of: trigger: detection of photoacoustic light with a photodiode; and data acquisition: 128 times (128 pulses) on average.

As a result of the measurement of the photoacoustic signal at a wavelength of 780 nm, the J-ICG-0.2 μm was found to generate a photoacoustic signal having an intensity 1.7 times as high as that of ICG. This is probably because the dye aggregates and a Gruneisen coefficient per unit dye increases. In addition, the J-ICG was found to be capable of generating a photoacoustic signal even at an absorption wavelength of 895 nm. The photoacoustic signal of ICG could not be detected because ICG had no absorption at 895 nm.

### Example 1-2

### Preparation of J-Aggregate of ICG Containing Phospholipid

20 Milliliters of distilled water were added to 23.4 mg of ICG. 47.8 Milligrams of a phospholipid DSPC were added to the mixture and then the whole was irradiated with an ultrasonic wave for 3 minutes to prepare a 1.5-mM aqueous solution of ICG containing DSPC. The aqueous solution of ICG was warmed in a dark place at 65° C. for 24 hours. Next, the aqueous solution of ICG containing DSPC was left at rest in a dark place at room temperature for 5 days to provide a J-aggregate of ICG containing DSPC. Hereinafter, the J-aggregate of ICG containing DSPC thus prepared is abbreviated as “J-ICG-DSPC.” An aqueous solution of the J-ICG-DSPC was kept in cold storage after the preparation.

(Filtration with Pore Filter)

The aqueous solution of the J-ICG-DSPC was filtered with a pore filter and then the filtrate was recovered. The pore size of the pore filter used here was 0.2 μm, and the J-ICG-DSPC obtained through the filtration with the pore filter is hereinafter abbreviated as “J-ICG-DSPC-0.2 μm.”

(Absorption Spectrum Measurement)

The absorbances at 780 nm and 895 nm of the resultant J-ICG-DSPC-0.2 μm was measured in the same manner as in Example 1-1. Table 5 shows the Abs895, the Abs780, and the Abs895/Abs780. As is apparent from Table 5, the presence of the J-aggregate of the J-ICG-DSPC-0.2 μm was able to be confirmed.

TABLE 5

| | Abs895 | Abs780 | Abs895/Abs780 |
|---|---|---|---|
| J-ICG-DSPC-0.2 μm | 0.46 | 0.12 | 3.8 |

(Particle Size Measurement)

The particle size of the resultant J-ICG-DSPC-0.2 μm was measured by the DLS method in the same manner as in Example 1-1. The J-ICG-DSPC-0.2 μm had a particle size of 178 nm and a polydispersity index of 0.24.

(Evaluation for Accumulation Ratio in Lymph Node)

In the same manner as in Example 1-1, the resultant J-ICG-DSPC-0.2 μm was subcutaneously administered to a planta of a mouse, and 24 hours after the administration, its accumulation ratio in a lymph node below the knee was measured. The accumulation ratio of the J-ICG-DSPC-0.2 μm in the lymph node was 0.97% and hence was observed to be reinforced so as to be 9.7 times as high as that of ICG.

(Fluorescent Imaging of Lymph Node)

In the same manner as in Example 1-1, the resultant J-ICG-DSPC-0.2 μm was subcutaneously administered to a planta of a mouse, and 24 hours after the administration, a lymph node below the knee was extirpated and subjected to fluorescent imaging. A fluorescence signal from the lymph node was confirmed.

### Example 1-3

### Preparation of J-Aggregate of ICG Containing Phospholipid HSPC

20 Milliliters of distilled water were added to 23.4 mg of ICG and then 4.8 mg, 23 mg, 47.8 mg, or 240 mg of hydrogenated soy phosphatidylcholine (HSPC, NC-21E, manufactured by NOF CORPORATION) were added to the mixture. Molar ratios (HSPC/ICG) of HSPC to ICG were 0.2, 1.0, 2.0, and 9.9, respectively. Each solution was irradiated with an ultrasonic wave for 3 minutes to prepare a 1.5-mM aqueous solution of ICG containing HSPC. A 1.5-mM aqueous solution of ICG to which HSPC had not been added (having an HSPC/ICG of 0) was also prepared as a control. The aqueous solution of ICG was warmed in a dark plate at 65° C. for 24 hours. Next, each aqueous solution of ICG containing HSPC was left at rest in a dark place at room temperature for 5 days to provide a J-aggregate of ICG containing HSPC. Hereinafter, the J-aggregates of ICG prepared at molar ratios (HSPC/ICG) of HSPC to ICG of 0, 0.2, 1.0, 2.0, and 9.9 are abbreviated as “J-ICG-HSPC-0,” “J-ICG-HSPC-0.2,” “J-ICG-HSPC-1.0,” “J-ICG-HSPC-2.0,” and “J-ICG-HSPC-9.9,” respectively. Those aqueous solutions were kept in cold storage after their preparation.

(Filtration with Pore Filter)

The aqueous solution of the J-ICG-HSPC was filtered with a pore filter and then the filtrate was recovered. The pore sizes of the pore filters used here were 0.2 μm and 0.1 μm, and respective J-ICG-HSPC's obtained through the filtration with the pore filters are hereinafter abbreviated as “J-ICG-DSPC-X-0.2 μm” and “J-ICG-DSPC-X-0.1 μm.” Here, X represents a molar ratio (HSPC/ICG) of HSPC to ICG. For example, a J-ICG-HSPC having a molar ratio (HSPC/ICG) of HSPC to ICG of 1.0 and obtained through the filtration with the pore filter having a pore size of 0.2 μm is abbreviated as “J-ICG-HSPC-1.0-0.2 μm.”

(Absorption Spectrum Measurement)

A ratio (Abs895/Abs780) of the absorbance of each of the resultant J-ICG-HSPC's at 780 nm to its absorbance at 895 nm and a ratio (Abs700/Abs780) of its absorbance at 700 nm to its absorbance at 780 nm were measured in the same manner as in Example 1-1. Here, the Abs895/Abs780 is a parameter representing the formation ratio of a J-aggregate and the Abs700/Abs780 is a parameter representing the formation ratio of an H-aggregate. Table 6 shows a relationship among a molar ratio (HSPC/ICG) of HSPC to ICG, the pore size of a pore filter, the Abs895/Abs780, and the Abs700/Abs780. As is apparent from Table 6, an increase in HSPC/ICG increased the formation ratio of the H-aggregate while reducing the formation ratio of the J-aggregate. In other words, a large amount of HSPC was found to have an inhibiting effect on the J-aggregation of ICG.

TABLE 6

| | | Molar ratio (HSPC/ICG) | | | | |
|---|---|---|---|---|---|---|
| | | of HSPC to ICG | | | | |
| | | 0.0 | 0.2 | 1.0 | 2.0 | 9.9 |
| 0.2 μm | Abs895/Abs780 | 6.4 | 6.0 | 6.0 | 5.0 | 0.6 |
| | Abs700/Abs780 | 0.3 | 0.5 | 0.5 | 0.6 | 0.7 |
| 0.1 μm | Abs895/Abs780 | 6.1 | 5.8 | 5.8 | 4.8 | 0.3 |
| | Abs700/Abs780 | 0.4 | 0.5 | 0.5 | 0.6 | 0.7 |

(Particle Size Measurement)

The particle sizes of the resultant J-ICG-HSPC's were measured by the DLS method in the same manner as in Example 1-1. Table 7 shows a relationship among a molar ratio (HSPC/ICG) of HSPC to ICG, the pore size of a pore filter, a particle size, and a polydispersity index. As is apparent from Table 7, an increase in HSPC/ICG reduced the particle size. Table 6 showed that while HSPC had an inhibiting effect on the J-aggregation of ICG, HSPC had a reducing effect on the particle size, and in the case of an HSPC/ICG of 9.9, a particle of 30 to 40 nm that was the smallest particle containing a J-aggregate of ICG was obtained. In addition, with regard to an effect of the pore size of a pore filter, the tendency that a particle filtered with the 0.1-μm pore filter had a smaller size than that of a particle filtered with the 0.2-μm pore filter was observed.

TABLE 7

| | | Molar ratio (HSPC/ICG) of HSPC to ICG | | | | |
|---|---|---|---|---|---|---|
| | | 0.0 | 0.2 | 1.0 | 2.0 | 9.9 |
| 0.2 μm | Particle size | 360 | 294 | 227 | 161 | 40 |
| | (nm) | | | | | |
| | Polydispersity | 0.25 | 0.24 | 0.28 | 0.43 | 0.44 |
| | index (PDI) | | | | | |
| 0.1 μm | Particle size | 293 | 260 | 207 | 138 | 32 |
| | (nm) | | | | | |
| | Polydispersity | 0.22 | 0.23 | 0.27 | 0.41 | 0.38 |
| | index (PDI) | | | | | |

(Evaluation for Stability of Aggregate in Serum)

Table 8 shows the results. It should be noted that in the case of an HSPC/ICG of 9.9, stability measurement could not be performed because no particle precipitated through centrifugal separation under the experimental condition. Table 8 revealed that while an increase in HSPC/ICG caused a slight reduction in stability, the aggregate showed extremely small dissociation into an ICG monomer and was hence sufficiently stable.

TABLE 8

| | Molar ratio (HSPC/ICG) of | | | | | ICG |
|---|---|---|---|---|---|---|
| | HSPC to ICG | | | | | (Comparative |
| | 0.0 | 0.2 | 1.0 | 2.0 | 9.9 | Example) |
| Stability | 0.87 | 0.85 | 0.83 | 0.72 | Unmeasurable | 0.19 |
| of | | | | | | |
| aggregate | | | | | | |
| (nm) | | | | | | |

The foregoing results showed that a J-aggregate of ICG containing HSPC had a particle size of the order of nanometers and was sufficiently stable even in a serum environment. Further, the results showed that the size was able to be controlled according to the amount of HSPC. The present invention was the first to enable the acquisition of a particle having a size as small as several tens of nanometers, which could not have been achieved by a conventional method, in a J-aggregate particle of ICG.

### Example 2

### Example 2-1

### Synthesis of J-Aggregated ICG-Containing Nanoparticle 1

ICG (4.4 mg, manufactured by the Society of Japanese Pharmacopoeia) was dissolved in 1 ml of methanol to prepare a methanol solution of ICG. DSPC (9 mg, manufactured by NOF CORPORATION) was dissolved in 1 ml of chloroform to prepare a chloroform solution of DSPC. One milliliter of the methanol solution of ICG and 1 ml of the chloroform solution of DSPC were mixed, and then the solvents were distilled off under reduced pressure at 40° C. ICG and DSPC evaporated to dryness were dissolved in 1.6 ml of chloroform to prepare an ICG composition 1 obtained by dissolving ICG and DSPC in chloroform.

Next, the ICG composition 1 was added to an aqueous solution (20 ml) prepared by dissolving the phospholipid represented by the chemical formula 2 (7.3 mg, DSPE-PEG-OCH₃, M.W of PEG: 2,000, manufactured by NOF CORPORATION) to provide a mixed liquid, and then the mixed liquid was stirred. After that, the liquid was treated with an ultrasonic disperser for 90 seconds to prepare an O/W type emulsion.

Next, chloroform was distilled off from the dispersoid by decompressing the emulsion with a rotary evaporator (at 40° C. for 2 hours). Thus, an aqueous dispersion liquid of an ICG-containing nanoparticle in which the surface of a fine particle was protected with the DSPE-PEG-OCH₃ was obtained. Hereinafter, the ICG-containing nanoparticle is referred to as “ICG_NP1.”

Further, the ICG-containing nanoparticle was incubated at 37° C. for 24 hours to provide an aqueous dispersion liquid 1 of a nanoparticle containing J-aggregated ICG. Hereinafter, the J-aggregated ICG-containing nanoparticle is referred to as “J-ICG_NP1.”

### Synthesis of J-Aggregated ICG-Containing Nanoparticle 2

An aqueous dispersion liquid of a nanoparticle was obtained by the same method as the foregoing except that the amount of DSPC was set to 18 mg. Hereinafter, the nanoparticles obtained at this time are referred to as “ICG_NP2” and “J-ICG_NP2,” respectively.

### Synthesis of J-Aggregated ICG-Containing Nanoparticle 3

An aqueous dispersion liquid of a nanoparticle was obtained by the same method as the foregoing except that the amount of DSPC was set to 27 mg. Hereinafter, the nanoparticles obtained at this time are referred to as “ICG_NP3” and “J-ICG_NP3,” respectively.

### Example 2-2

### Evaluation for Absorption Spectrum of J-Aggregated ICG-Containing Nanoparticle

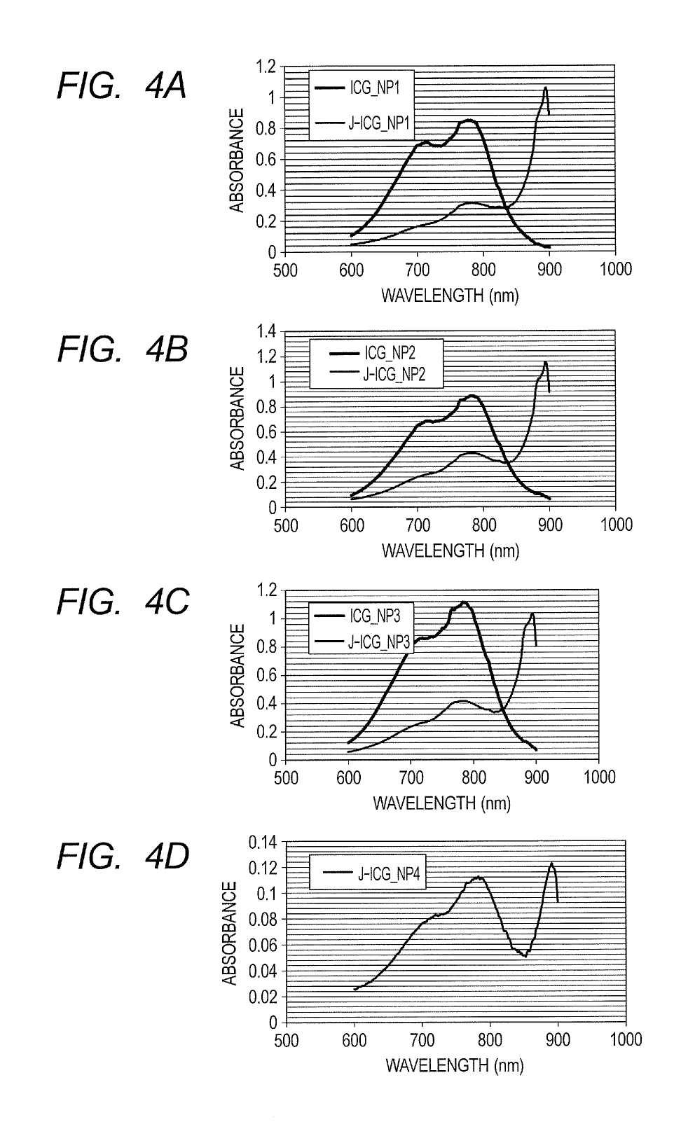

The absorption spectra of the ICG_NP's and the J-ICG_NP's were measured. As a reference, an aqueous solution of ICG was similarly subjected to the measurement. FIG. 4A, FIG. 4B, and FIG. 4C each show the absorption spectra of an ICG_NP and a J-ICG_NP. As is apparent from FIG. 4A, FIG. 4B, and FIG. 4C, an absorption maximum near 895 nm derived from the J-aggregate of ICG was present and hence the presence of the J-aggregate was able to be confirmed.

### Example 2-3

### Particle Size Measurement

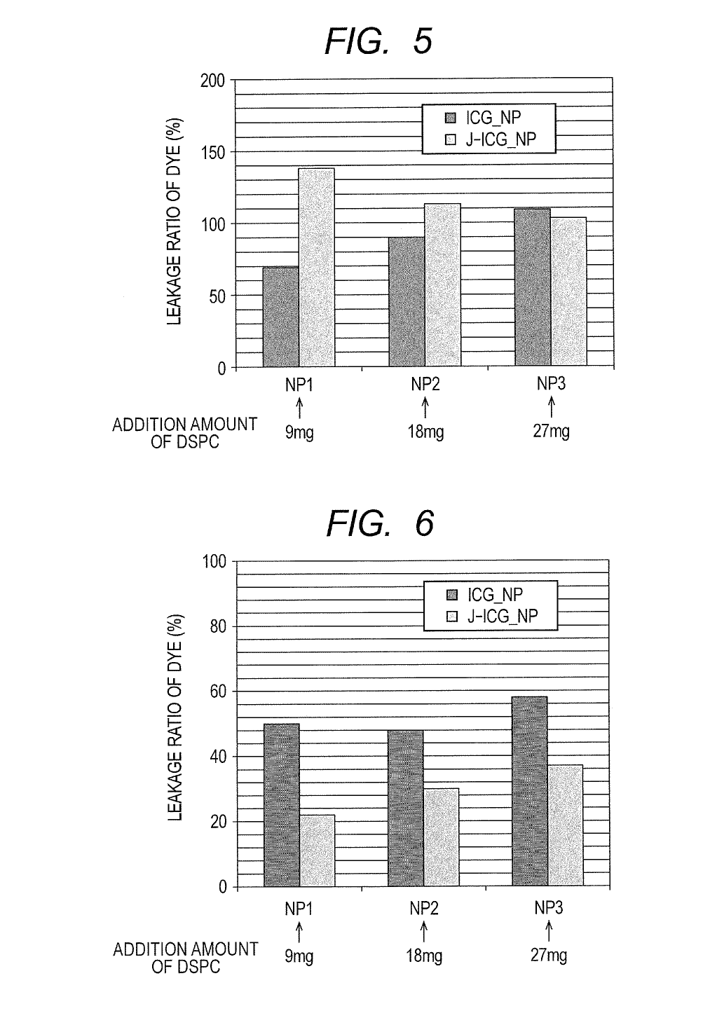

The particle sizes of the ICG-containing nanoparticles (ICG_NP's) and the J-aggregated ICG-containing nanoparticles (J-ICG_NP's) were analyzed with a dynamic light scattering analyzer (manufactured by Otsuka Electronics Co., Ltd., DLS-8000). Table 9 shows a mean particle size and polydispersity index (PDI) obtained in the foregoing. It was able to be confirmed that the J-aggregated ICG-containing nanoparticles each retained a mean particle size of 200 nm or less despite the fact that ICG formed the J-aggregate. Further, as is apparent from FIG. 5, a tendency was observed that the particle size of a particle containing the J-aggregate of ICG reduced as the addition amount of DSPC increased.

The J-aggregate of ICG reported heretofore is generally a polydispersion having a mean particle size of about several microns and a wide particle size distribution. On the other hand, it was able to be confirmed that the J-aggregated ICG-containing nanoparticles obtained in this example each had a mean particle size of 200 nm or less and the polydispersity indices of the particles were small. Further, it was suggested that the particle sizes of the J-aggregated ICG-containing nanoparticles were controlled depending on the addition amount of DSPC.

The DLS method confirmed that the J-aggregated ICG-containing nanoparticles obtained by this example were J-aggregated ICG-containing nanoparticles whose particle sizes fell within the particle size range of 200 nm or less suitable for imaging.

TABLE 9

| | Particle size | PDI |
|---|---|---|
| ICG_NP1 | 69 nm | 0.157 |
| J-ICG_NP1 | 138 nm | 0.205 |
| ICG_NP2 | 90 nm | 0.159 |
| J-ICG_NP2 | 113 nm | 0.301 |
| ICG_NP3 | 109 nm | 0.201 |
| J-ICG_NP3 | 103 nm | 0.220 |

### Example 2-4

### Evaluation for Stability of Aggregate in Serum

The ICG_NP's and the J-ICG_NP's were each evaluated for the stability of the aggregate in a serum in a fetal bovine serum (FBS) by the same method as that of Example 1, provided that as is apparent from FIG. 6, it was able to be confirmed that causing ICG of each ICG-containing nanoparticle to form the J-aggregate significantly reduced the leakage of the dye in the serum.

### Example 2-5

### Composition of Each of ICG and DSPC in J-Aggregated ICG-Containing Nanoparticle

The composition of each of ICG and DSPC in each of the particles J-ICG_NP's was calculated. The amount of ICG, the amount of DSPC, and a particle weight were calculated by the following methods.

The amount of ICG: The calibration curve of a concentration and an absorbance was produced by dissolving various concentrations of ICG in 90% DMF in advance. After that, a sample was dissolved in 90% DMF and then its absorbance was measured by the method, followed by the calculation of the amount of ICG at that time.

Amount of DSPC: The amount of DSPC in the sample was measured with a Phospholipid C-Test Wako. The amount of DSPC was calculated according to an accompanying operation method.

Particle weight: The particle weight of the sample was calculated by a freeze-drying method.

Table 10 shows the resultant composition (wt %) of each of ICG and DSPC. Those results were able to confirm that a ratio of ICG and DSPC to the particle weight was 30 wt % or more.

TABLE 10

| | | DSPC |
|---|---|---|
| | ICG [%] | [%] |
| J-ICG_NP1 | 2 | 49 |
| J-ICG_NP2 | 3 | 49 |
| J-ICG_NP3 | 4 | 38 |

### Example 2-6

### Synthesis of J-Aggregated ICG-Containing Nanoparticle Using No Surfactant and Evaluations for its Physical Properties

An aqueous dispersion liquid of a J-aggregated ICG-containing nanoparticle was obtained by the same method as the foregoing except that the amount of DSPC was set to 27 mg and no surfactant was added to the aqueous solution. Hereinafter, the aqueous dispersion liquid is referred to as “J-ICG_NP4.” The absorption spectrum measurement and particle size measurement of the J-ICG_NP4 were performed by the methods. As is apparent from FIG. 4D showing the absorption spectrum of the J-ICG_NP4 and Table 11 showing its particle size, it was able to be confirmed that the J-ICG_NP4 was constituted of J-aggregated ICG having a particle size of 200 nm or less.

TABLE 11

| | Particle size | PDI |
|---|---|---|
| J-ICG_NP4 | 136 nm | 0.201 |

### Example 2-7

### Photoacoustic Measurement of J-Aggregated ICG-Containing Nanoparticle

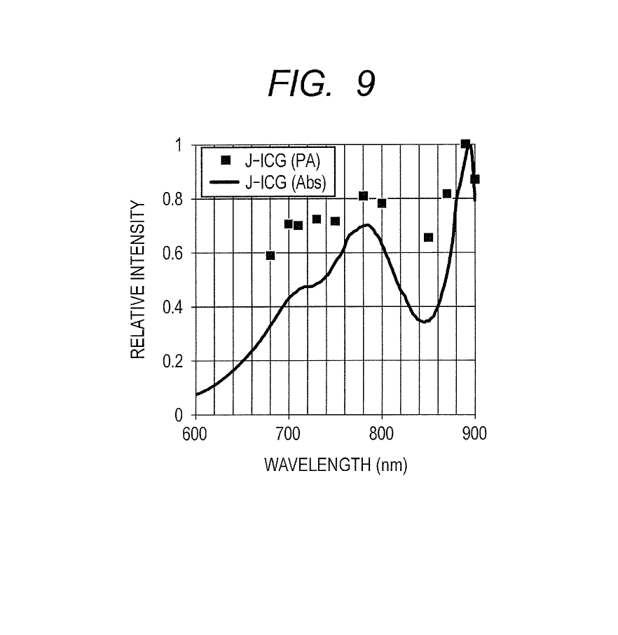

The J-ICG_NP1 produced by the method of Example 2-1 was subjected to a photoacoustic spectrum evaluation. Photoacoustic signal measurement was performed with a commercial photoacoustic imaging apparatus (Nexus 128, Endra Inc.). A tube made of a polyethylene (having an inner diameter of 1 mm) was used as a measurement vessel. The J-ICG_NP1 was diluted with a fetal bovine serum (FBS) so as to have a dye concentration of 10 μM and then filled into the tube, followed by the performance of the photoacoustic measurement. A photoacoustic signal in a region of interest (ROI) was acquired from the resultant photoacoustic data with an image analysis software (Micro VIEW, GE Healthcare). FIG. 9 shows the relative absorption spectrum (an absorption maximum in a measuring range is set to 1) and photoacoustic relative intensity (the highest photoacoustic intensity at a measurement wavelength is set to 1) of the J-ICG_NP1. As is apparent from the results, it was able to be confirmed that the absorption maximum appeared near 900 nm through the transformation of ICG into the J-aggregate and the intensity of the photoacoustic signal near 900 nm increased in association with the appearance.

### Example 3

### Example 3-1

### Preparation (1) of Liposome Encapsulating ICG

95.8 Milligrams of hydrogenated soy phosphatidylcholine (HSPC, NC-21E, manufactured by NOF CORPORATION), 31.9 mg of a distearoyl phosphatidyl ethanolamine polyethylene glycol (DSPE-PEG, manufactured by NOF CORPORATION), and 31.9 mg of cholesterol were dissolved in 10 mL of chloroform. 11.7 Milligrams of ICG were added to the solution and then the mixture was irradiated with an ultrasonic wave for 5 minutes (three-frequency ultrasonic cleaner VS-100III, AS ONE Corporation, 28 kHz). Next, the solution was transferred to an eggplant flask and then chloroform was distilled off at 40° C. under reduced pressure, followed by the performance of vacuum drying overnight. Ten milliliters of a 10-mM HEPES solution (having a pH of 7.2) were added to the resultant dried and hardened product of the lipids and ICG, and then the mixture was irradiated with an ultrasonic wave. The ultrasonic irradiation was performed for 30 minutes as a whole by repeating the following cycle: at 28 kHz for 10 seconds, at 45 kHz for 10 seconds, and at 100 kHz for 10 seconds. After that, the mixture was irradiated with an ultrasonic wave at 28 kHz for an additional thirty minutes. A temperature at the time of the ultrasonic irradiation was set to 60° C. After that, an ultrasonic treatment was performed for 15 minutes with a probe type ultrasonic irradiation apparatus. After that, the treated product was filtered with a syringe filtration filter having a pore size of 0.2 μm. Hereinafter, the resultant liposome is referred to as “JIL1.” The HEPES solution of the JIL1 was stored at 4° C.

### Example 3-2

### Preparation (2) of Liposome Encapsulating J-Aggregate of ICG

The HEPES solution of the JIL1 obtained in Example 3-1 was warmed under a light-shielding condition at each of 4° C., 37° C., and 65° C. A warming time was set to 17 hours, and after the warming, the solution was filtered with a syringe filtration filter having a pore size of 0.2 μm. The resultant solution was stored at 4° C. Hereinafter, the resultant liposomes each encapsulating the J-aggregate of ICG are abbreviated as “JIL1-4,” “JIL1-37,” and “JIL1-65,” respectively.

### Example 3-3

### Absorption Spectrum Measurement

The absorbances at 780 nm and 895 nm of the JIL1-4, JIL1-37, and JIL1-65 obtained in Example 3-2 were measured. Absorption at 780 nm is derived from the monomer of ICG and that at 895 nm is derived from the J-aggregate. That is, a ratio (sometimes abbreviated as “Abs895/Abs780”) of the absorbance at 895 nm (sometimes abbreviated as “Abs895”) to the absorbance at 780 nm (sometimes abbreviated as “Abs780”) may represent the formation ratio of the J-aggregate. Table 12 shows the Abs895, Abs780, and Abs895/Abs780 of each aqueous solution. It should be noted that in the measurement, a quartz cell having an optical path length of 1 cm was used and an aqueous solution prepared by diluting the liposome solution obtained in Example 3-2 with distilled water 200-fold was used. As is apparent from Table 12, in each of all the samples, absorption appeared at 895 nm and hence the formation of the J-aggregate of ICG was observed. The formation of the J-aggregate was promoted as the temperature increased. Surprisingly, the formation of the J-aggregate was observed even in the JIL1-4 that had not been warmed. The ultrasonic treatment at the time of the preparation of the liposome may serve as a trigger for aggregate formation.

TABLE 12

| | Abs895 | Abs780 | Abs895/Abs780 |
|---|---|---|---|
| JIL1-4 | 0.21 | 0.68 | 0.31 |
| JIL1-37 | 0.44 | 0.68 | 0.65 |
| JIL1-65 | 0.67 | 0.56 | 1.20 |

### Example 3-4

### Particle Size Measurement

The particle sizes of the JIL1-4, JIL1-37, and JIL1-65 obtained in Example 3-2 were measured by the DLS method. As is apparent from Table 13, all the samples each had a particle size of about 100 nm. It has been known that a J-aggregate of ICG has a mean particle size of several micrometers. However, a J-aggregate of ICG that has a particle size of 100 nm and is relatively monodisperse has not been reported so far. The liposome of the present invention enabled the success of the formation of the J-aggregate of ICG in the nanosize environment of the liposome.

TABLE 13

| | Cumulant particle size | Polydispersity index |
|---|---|---|
| | (nm) | (PDI) |
| JIL1-4 | 104.2 | 0.27 |
| JIL1-37 | 107.8 | 0.27 |
| JIL1-65 | 101.5 | 0.29 |

### Example 3-5

### Purification of Liposome By Centrifugation

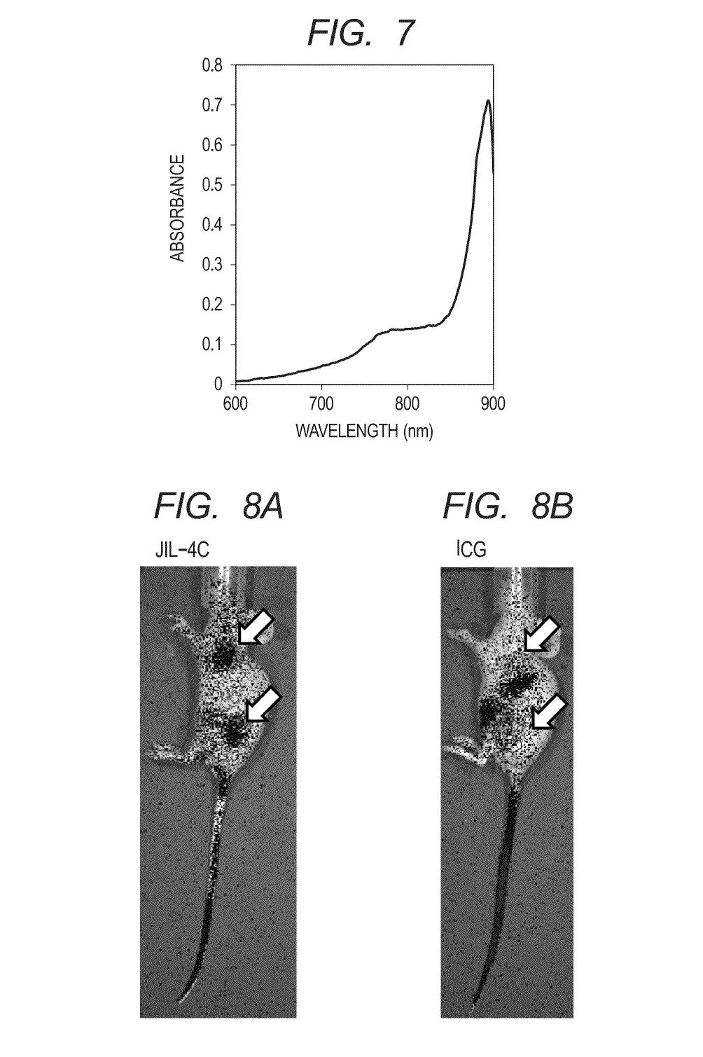

The JIL1-4 obtained in Example 3-2 was subjected to ultracentrifugation (280,000×g, room temperature, 17 minutes) and then the precipitate was recovered. The precipitate was resuspended in a 10-mM HEPES solution again and then the suspension was subjected to ultracentrifugation (280,000×g, room temperature, 17 minutes), followed by the recovery of the precipitate. The precipitate was resuspended in a 10-mM HEPES solution, and then the supernatant after centrifugation (20,000×g, room temperature, 5 minutes) was recovered and filtered with a syringe filtration filter having a pore size of 0.2 μm. Hereinafter, the resultant liposome is referred to as “JIL1-4C.” The particle size of the JIL1-4C was measured by the DLS method. As a result, the JIL1-4C had a particle size of 102.5 nm and a polydispersity index (PDI) of 0.11. It is assumed that the phospholipid, ICG, and the J-aggregate of ICG as impurities were removed by the centrifugation, and as a result, the PDI reduced, i.e., the particle size distribution narrowed. FIG. 7 shows the absorption spectrum of the JIL1-4C. The Abs895/Abs780 was 4.5. In addition, the zeta potential of the JIL1-4C was measured in 10-mM HEPES at a pH of 7.4. As a result, the zeta potential was −55.1 mV.

### Example 3-6

### Evaluation for ICG Encapsulation Ratio in Serum

A serum solution was used as an in vivo model for measuring the ICG encapsulation ratio of the JIL1-4C obtained in Example 3-5 in a living organism. In other words, the ICG encapsulation ratio of the JIL1-4C in the serum was evaluated. A method for the evaluation is as described below. First, 0.1 mL of an aqueous solution of the JIL1-4C was transferred to a sample tube and then 0.9 mL of a bovine serum was added to the solution. Next, the mixture was incubated in a dark plate at 37° C. for 24 hours. Next, the Abs800 of the JIL1-4C was measured. Next, the sample was subjected to ultracentrifugation (280,000 G, 17 minutes, room temperature, sample volume: 0.5 mL) to precipitate the JIL1-4C. After the centrifugation, the Abs800 of the supernatant was measured. It has been confirmed that ICG does not precipitate under the centrifugation conditions. Therefore, the ICG encapsulation ratio in the serum is defined as described below.

ICG encapsulation ratio (%) in serum=1-(Abs800 of centrifugation supernatant/Abs800 before centrifugation)

The ICG encapsulation ratio of the JIL1-4C in the serum was 54.9%.

### Example 3-7

### Evaluation for Accumulation Ratio in Lymph Node

The JIL1-4C obtained in Example 3-5 (13 nmol in terms of ICG) was subcutaneously administered to a planta of a mouse, and 24 hours after the administration, its accumulation ratio in a lymph node below the knee was measured. As a comparative example, the aqueous solution of ICG was similarly subjected to the measurement. 24 Hours after the administration, the lymph node below the knee was extirpated and then the extirpated lymph node was homogenized with a 1% Triton-X100 aqueous solution. The amount (mol) of the dye that had accumulated in the lymph node was determined by measuring the Abs800 of the solution. Next, the accumulation ratio (%) in the lymph node was calculated by dividing the value by 13 nmol as the administration amount and multiplying the resultant by 100.

As is apparent from Table 14, the accumulation ratio of the JIL1-4C in the lymph node increased so as to be 12 times as high as that of ICG. The result showed the effectiveness of the JIL1-4C as a contrast agent for a lymph node.

TABLE 14

| | Accumulation ratio in lymph node (%) |
|---|---|
| JIL1-4C | 1.2 |
| ICG | 0.1 |

### Example 3-8

### Fluorescent Imaging of Lymph Node

The lymph node below the knee of the mouse to the planta of which the JIL1-4C had been subcutaneously administered obtained in the experiment of Example 3-7 was extirpated and then subjected to fluorescent imaging. A fluorescence signal from the extirpated lymph node was confirmed. The result showed the effectiveness of the JIL1-4C as a fluorescent contrast agent for a lymph node.

### Example 3-9

### Confirmation of Tumor-Contrasting Ability of Liposome

The JIL1-4C obtained in Example 3-5 was evaluated for its tumor-contrasting ability. The fluorescent imaging of a cancer-bearing mouse to which the JIL1-4C had been administered was performed. In the fluorescent imaging experiment, female outbred BALB/c Slc-nu/nu mice (six-week old at the time of purchase) (Japan SLC, Inc.) were used. For 1 week before the causing of the mice to bear cancers, the mice were habituated through the use of a normal diet and bed in the animal facility of Kyoto University, Faculty of Medicine (Kyoto Prefecture, Japan) under such an environment that the diet and drinking water were available ad libitum. About 2 weeks before the imaging experiment, 2×10⁶ N87 human stomach cancer cells (ATCC#CRL-5822) were subcutaneously injected into the shoulders and femurs of the mice. The mice were compared after having been classified into two groups, i.e., the JIL1-4C according to the present invention and ICG as a control. The administration amount was 13 nmol per mouse in terms of a dye amount and the dye was injected as 100 μL of a PBS solution into the tail vein of each mouse. With regard to the whole-body fluorescence image of the mouse to which a probe had been administered, the bright-field image and fluorescence image of the mouse were acquired with an IVIS (trademark) Imaging System 200 Series (XENOGEN) 24 hours after the administration. FIG. 8A and FIG. 8B are the fluorescence images of the mice 24 hours after the administration. FIG. 8A shows the mouse to which the JIL1-4C was administered and a fluorescence signal at a cancer-bearing site indicated by a white arrow was confirmed. FIG. 8B shows the mouse to which ICG was administered as a control and a fluorescence signal at a cancer-bearing site indicated by a white arrow could not be confirmed. Signals from a liver and an intestine were confirmed. The results showed that the JIL1-4C was able to contrast a tumor and showed its effectiveness as a tumor contrast agent.

### Example 3-10

### Confirmation of Accumulation of Liposome in Tumor

The amount of the dye in the cancer tissue of a mouse of the tumor imaging experiment performed in Example 3-9 was determined. First, the mouse was euthanized with a carbon dioxide gas 24 hours after the administration, followed by the extirpation of the cancer tissue. The cancer tissue was transferred to a plastic tube and then homogenized by adding a 1% Triton-X100 aqueous solution in an amount 1.25 times as large as the weight of the cancer tissue. Next, dimethyl sulfoxide (DMSO) was added in an amount 20.25 times as large as the weight of the cancer tissue. The amount of the dye in the cancer tissue was determined by measuring the fluorescence intensity of the homogenized solution, which was in a state of being stored in the plastic tube, with an IVIS (trademark) Imaging System 200 Series (XENOGEN). As a result, no dye was detected in the cancer tissue of the ICG-administered mouse as a control, but the dye was detected in the cancer tissue of the JIL1-4C-administered mouse and the ratio of the transfer of the dye to the cancer tissue with respect to the administration amount was 1.7% (per 1 g of the cancer tissue).

### Example 3-11

### Preparation of Liposome Encapsulating ICG that has not J-Aggregated and Evaluation for ICG Encapsulation Ratio in Serum

In order for a liposome encapsulating ICG that had not J-aggregated to be prepared, a liposome was prepared according to Example 3-1, provided that the ultrasonic irradiation after the solvent removal of chloroform under reduced pressure was performed under ice cooling and then the purification described in Example 3-5 was immediately performed. The resultant liposome is referred to as “JIL1-Ctrl.” The particle size of the JIL1-Ctrl was measured by the DLS method. As a result, the JIL1-Ctrl had a particle size of 127.7 nm and a polydispersity index (PDI) of 0.15. The Abs895/Abs780 of the JIL1-Ctrl was 0.09 and it was confirmed that ICG had not J-aggregated. Immediately after the purification, an evaluation for an ICG encapsulation ratio in a serum was performed in the same manner as in Example 3-6. As a result, the ICG encapsulation ratio of the JIL1-Ctrl in the serum was 42.7%. As described in Examples 3-5 and 3-6, the JIL1-4C of the present invention had a particle size of 102.5 nm, a polydispersity index (PDI) of 0.11, and an ICG encapsulation ratio in the serum of 54.9%. As a result of comparison with the JIL1-Ctrl, it was found that the J-aggregation of ICG increased the ICG encapsulation ratio of the liposome in the serum. In addition, the J-aggregation reduced the particle size and improved dispersibility.

### Example 4

An example of the preparation of a liposome encapsulating J-aggregated ICG by a pH-gradient method is described below.

### Example 4-1

### Encapsulation in Liposome Utilizing pH Gradient (1) Preparation of Empty Liposome

Ten kinds of empty liposome dispersion liquids shown in Table 15 were prepared by changing the kinds of an internal aqueous phase (solution A) and an external aqueous phase (solution B). Distearoyl phosphatidylcholine (DSPC, COATSOME MC-8080, manufactured by NOF CORPORATION), cholesterol (manufactured by Wako Pure Chemical Industries, Ltd.), and a distearoyl phosphatidyl ethanolamine polyethylene glycol (DSPE-PEG, manufactured by NOF CORPORATION) were weighed at a weight ratio of 3:1:1. A total amount of 102 mg of the three kinds of lipids were dissolved in 2 ml of a mixed solution of methanol and chloroform (1:1), and then the solution was stirred at 37° C. for 1 hour. After that, the solvents were distilled off and then the remainder was vacuum-dried at room temperature overnight. Ten milliliters of each of the solutions A shown in Table 15 were added to the dried and hardened product of the lipids thus obtained (per a total amount of 102 mg of the three kinds of lipids), and then the mixture was stirred at 37° C. for 1 hour. Then, the mixture was subjected to an ultrasonic treatment (irradiated with an ultrasonic wave according to a cycle “28 kHz for 60 seconds→45 kHz for 60 seconds→100 kHz for 3 seconds”) at 60° C. for 30 minutes with a bath type ultrasonic apparatus (three-frequency ultrasonic cleaner VS-100III, manufactured by AS ONE Corporation). After that, the treated product was subjected to an extruder treatment at 65° C. (LIPEX 100 mL Thermobarrel Extruder, manufactured by Northern Lipids Inc.) and then passed through a membrane having a pore size of 100 nm. The particle size of the empty liposome dispersion liquid that had passed the membrane was measured by the dynamic light scattering method (DLS, Zeta-Sizer Nano, manufactured by Malvern Instruments). Table 15 shows the results. After that, the external aqueous phases of the empty liposome dispersion liquids were each replaced with the corresponding solution B shown in Table 15 by ultrafiltration (stirring type cell, Ultrafiltration Membrane 300 KDa, manufactured by Millipore Corporation), followed by concentration. Thus, the total lipid concentration of DSPC and cholesterol was adjusted to 40 mg/mL. The quantitative determination of DSPC and cholesterol was performed with a commercial determination kit (Phospholipid C-Test Wako, Cholesterol E-Test Wako, manufactured by Wako Pure Chemical Industries, Ltd.), provided that a mixture obtained by mixing an empty liposome liquid properly diluted with an HEPES buffer solution and a 4% aqueous solution of sodium dodecyl sulfate (SDS, manufactured by KISHIDA CHEMICAL Co., Ltd.) at 1:1 was subjected to the quantitative determination after having been warmed at 100° C. for 5 minutes.

TABLE 15

| | Solution A (internal | | | Solution B (external | | Particle size before | |
|---|---|---|---|---|---|---|---|
| | aqueous phase) | | | aqueous phase) | | replacement of external | |
| | HEPES | | | Citric acid | HEPES | aqueous phase | |
| | (pH 7.3) | Dextran | NaCl | (pH 3.0) | (pH 7.3) | Particle | Polydispersity |
| | mM | mg/ml | mM | mM | mM | size nm | index |
| Empty | 10 | 0 | 0 | 10 | 0 | 91 | 0.152 |
| liposome 1 | | | | | | | |
| Empty | 10 | 130 | 0 | 10 | 0 | 119 | 0.172 |
| liposome 2 | | | | | | | |
| Empty | 10 | 0 | 150 | 10 | 0 | 112 | 0.076 |
| liposome 3 | | | | | | | |
| Empty | 10 | 0 | 150 | 0 | 10 | | |
| liposome 4 | | | | | | | |
| Empty | 10 | 130 | 150 | 10 | 0 | 122 | 0.141 |
| liposome 5 | | | | | | | |
| Empty | 10 | 130 | 150 | 0 | 10 | | |
| liposome 6 | | | | | | | |
| Empty | 10 | 0 | 300 | 10 | 0 | 123 | 0.086 |
| liposome 7 | | | | | | | |
| Empty | 10 | 0 | 300 | 0 | 10 | | |
| liposome 8 | | | | | | | |
| Empty | 10 | 130 | 300 | 10 | 0 | 123 | 0.088 |
| liposome 9 | | | | | | | |
| Empty | 10 | 130 | 300 | 0 | 10 | | |
| liposome 10 | | | | | | | |

*0.308-M sucrose is added to each of all the solutions B.

HEPES (manufactured by Invitrogen) is a buffer solution having a concentration of 10 mM (a pH of 7.3). Dextran 40 (manufactured by Tokyo Chemical Industry Co., Ltd.) was used as the dextran. Citric acid was a buffer solution prepared to have a pH of 3.0 by dissolving citric acid monohydrate (manufactured by NACALAI TESQUE, LTD.) and trisodium citrate dihydrate (manufactured by NACALAI TESQUE, LTD.), and was used at a concentration of 10 mM.

### Example 4-2

### Encapsulation in Liposome Utilizing pH Gradient (2) Encapsulation and Purification of ICG

Indocyanine green (ICG, manufactured by the Society of Japanese Pharmacopoeia) was dissolved in the solutions B to produce ICG solutions each having an ICG concentration of 6 mg/ml, 2 mg/ml, or 0.1 mg/ml. Liposomes subjected to an ICG encapsulation treatment were prepared under 30 kinds of conditions by adding each of the ICG solutions having three concentrations to each of the ten kinds of empty liposome dispersion liquids. A preparation method under one condition is described below. The same operations were performed under any other condition as well. An ICG solution and an empty liposome dispersion liquid were placed in a thermostat at 60° C. for 15 minutes to be warmed to 60° C. 2.5 Milliliters of the ICG solution were added to 2.5 mL of the empty liposome dispersion liquid and then the mixture was stirred at 60° C. for 30 minutes. After that, 15 ml of the solution B were added thereto. The resultant was subjected to an extruder treatment at 65° C. and then passed through a membrane having a pore size of 50 or 80 nm. A liposome after the extruder treatment was recovered in a beaker cooled with ice.

While the solution B was injected, the recovered liposome dispersion liquid was purified by removing free ICG through an ultrafiltration treatment (Ultrafiltration Membrane 300 KDa). After the purification, the liquid was concentrated to an amount about one tenth of that before the purification and then recovered.

### Example 4-3

### Absorption Spectrum Measurement

The 30 kinds of liposomes subjected to the ICG encapsulation treatment obtained in Example 4-2 were each properly diluted with the HEPES buffer solution. The absorbances of each of the diluted solutions at wavelengths between 500 and 1,000 nm were measured, and then the maximum absorption wavelength and Abs895/Abs780 ratio thereof were determined. Thus, the formation of the J-aggregate in the liposome was confirmed.

Table 16 shows the results. Liposomes (p1-2, p1-3, p2-2, p2-3, p3-2, p3-3, p5-2, p5-3, p7-2, p7-3, p9-2, and p9-3), each of which was provided with a pH gradient and prepared by using a citric acid buffer solution in its external aqueous phase, each had an Abs895/Abs780 ratio as high as 2 or more and each showed a local maximum of an absorbance between 880 nm and 910 nm, and hence it was found that a liposome encapsulating J-aggregated ICG was able to be prepared. It should be noted that when liposomes (p1-1, p2-1, p3-1, p5-1, p7-1, and p9-1) were prepared at an addition concentration of ICG of 0.1 mg/ml, each of the liposomes had a low Abs895/Abs780 ratio and did not show a local maximum of an absorbance between 880 nm and 910 nm, and hence it was found that the concentration of added ICG needed to be increased to more than 0.1 mg/ml for causing ICG to form a J-aggregate.

On the other hand, liposomes (p4-1, p4-2, p4-3, p6-1, p6-2, p6-3, p8-1, p8-2, p8-3, p10-1, p10-2, and p10-3), each of which was provided with no pH gradient and prepared by using the HEPES buffer solution in its external aqueous phase, each had an Abs895/Abs780 ratio as low as 0.2 or less. In addition, none of the liposomes showed a maximum absorption wavelength around 895 nm and hence it was found that ICG in the liposome formed nearly no J-aggregate. It was found that the pH gradient needed to be provided for preparing a liposome encapsulating J-aggregated ICG.

Further, the addition of dextran 40 or sodium chloride (NaCl) to the internal aqueous phase was found to be capable of increasing the Abs895/Abs780 ratio.

### Example 4-4

### Particle Size Measurement

The particle sizes of the 30 kinds of liposomes subjected to the ICG encapsulation treatment obtained in Example 4-2 were measured by DLS (Zeta-Sizer Nano, manufactured by Malvern Instruments).

Table 16 shows the results. Each of the J-aggregated ICG-encapsulating liposomes was found to be capable of being prepared so as to have a particle size around 100 nm.

### Example 4-5

### Quantitative Determination of ICG, and Measurement of ICG Content and Molar Absorption Coefficient

The 30 kinds of liposomes subjected to the ICG encapsulation treatment obtained in Example 4-2 were subjected to the following assay evaluations.

Quantitative determination of ICG: A liposome liquid properly diluted with the HEPES buffer solution and dimethyl sulfoxide (DMF) were mixed at 1:9, and then the absorbance of the mixture at an OD of 790 nm was measured. In addition, an ICG standard solution dissolved in the HEPES buffer solution was similarly mixed with DMF and then the absorbance of the mixture at an OD of 790 nm was measured. A standard curve was produced based on the measured values and then the concentration of ICG in the liposome was determined.

ICG content: The ratio of the amount of ICG in the dry weight of the liposome was calculated.

Molar absorption coefficient: An absorbance (optical path length: 1 cm) at the maximum absorption wavelength in a liposome particle concentration of 1 M was measured. The liposome particle concentration was determined as described below. A weight per one liposome was calculated from the volume of the liposome, which was calculated from the mean particle size of the liposome, by regarding its specific gravity as 1. The concentration of the liposome was calculated from the weight of the liposome and the dry weight of the liposome.

Table 16 shows the results. It was found that the ICG concentration, the ICG content, and the molar absorption coefficient increased as the Abs895/Abs780 ratio increased. It was found that when ICG was encapsulated in a liposome, a method involving introducing ICG while causing ICG to form a J-aggregate through the application of a pH gradient was able to increase the ICG content in the liposome and hence was effective for the preparation of a liposome type contrast agent for photoacoustic imaging having a high molar absorption coefficient.

### Example 4-6

### Quantitative Determination of DSPC

A liposome liquid properly diluted with the HEPES buffer solution and a 4% aqueous solution of SDS were mixed at 1:1, and then the mixture was warmed at 100° C. for 5 minutes, followed by measurement with a phospholipid determination kit (Phospholipid C-Test Wako). In addition, its DSPC content was calculated from the ratio of the amount of DSPC in the dry weight of the liposome. Table 16 shows the results. It was able to be confirmed that the ratio of DSPC to the particle weight was 30 wt % or more in each of all samples.

TABLE 16

| | Sample name | | | | | | | | | | | | | | |
|---|---|---|---|---|---|---|---|---|---|---|---|---|---|---|---|
| | p1-1 | p1-2 | p1-3 | p2-1 | p2-2 | p2-3 | p3-1 | p3-2 | p3-3 | p4-1 | p4-2 | p4-3 | p5-1 | p5-2 | p5-3 |
| Kind of empty | Empty liposome 1 | | | Empty liposome 2 | | | Empty liposome 3 | | | Empty liposome 4 | | | Empty liposome 5 | | |
| liposome | | | | | | | | | | | | | | | |
| Internal | HEPES | | | HEPES + dextran 130 mg/mL | | | HEPES + NaCl 150 mM | | | HEPES + NaCl 150 mM | | | HEPES + dextran 130 mg/mL + | | |
| aqueous phase | | | | | | | | | | | | | NaCl 150 mM | | |
| External | Citric acid + 0.308M | | | Citric acid + 0.308M | | | Citric acid + 0.308M | | | HEPES + 0.308M sucrose | | | Citric acid + 0.308M | | |
| aqueous phase | sucrose | | | sucrose | | | sucrose | | | | | | sucrose | | |
| Concentration | 0.1 | 2 | 6 | 0.1 | 2 | 6 | 0.1 | 2 | 6 | 0.1 | 2 | 6 | 0.1 | 2 | 6 |
| of added ICG | | | | | | | | | | | | | | | |
| mg/ml | | | | | | | | | | | | | | | |
| OD (780 nm) | 6.9 | 169.2 | 339.2 | 8.0 | 94.4 | 273.6 | 5.3 | 92.0 | 205.2 | 11.1 | 47.8 | 51.0 | 2.6 | 128.8 | 342.0 |
| OD (895 nm) | 0.8 | 440.8 | 1641.6 | 3.7 | 460.8 | 1690.8 | 2.6 | 472.0 | 1238.4 | 0.8 | 3.8 | 3.4 | 0.4 | 614.4 | 2058.0 |
| 895/780 ratio | 0.11 | 2.61 | 4.84 | 0.46 | 4.88 | 6.18 | 0.49 | 5.13 | 6.04 | 0.07 | 0.08 | 0.07 | 0.17 | 4.77 | 6.02 |
| Maximum | 795 | 891 | 893 | 792 | 891 | 892 | 787 | 891 | 893 | 802 | 804 | 804 | 791 | 893 | 893 |
| absorption | | | | | | | | | | | | | | | |
| wavelength | | | | | | | | | | | | | | | |
| Particle size | 97 | 102 | 101 | 108 | 106 | 106 | 92 | 119 | 107 | 113 | 139 | 105 | 89 | 104 | 106 |
| (nm) | | | | | | | | | | | | | | | |
| Polydispersity | 0.209 | 0.203 | 0.221 | 0.154 | 0.036 | 0.114 | 0.146 | 0.142 | 0.122 | 0.275 | 0.363 | 0.194 | 0.091 | 0.087 | 0.094 |
| index (PDI) | | | | | | | | | | | | | | | |
| ICG | 0.05 | 1.27 | 3.01 | 0.02 | 0.81 | 2.62 | 0.02 | 0.81 | 2.06 | 0.06 | 0.34 | 0.35 | 0.02 | 1.13 | 3.45 |
| concentration | | | | | | | | | | | | | | | |
| (mg/mL) | | | | | | | | | | | | | | | |
| ICG content | 0.1 | 2.3 | 6.2 | 0.1 | 2.8 | 7.7 | 0.1 | 3.2 | 6.8 | 0.2 | 1.0 | 1.2 | 0.1 | 2.6 | 8.3 |
| (%) | | | | | | | | | | | | | | | |
| DSPC | 24 | 26 | 25 | 13 | 16 | 17 | 19 | 17 | 14 | 19 | 25 | 18 | 16 | 19 | 19 |
| concentration | | | | | | | | | | | | | | | |
| (mg/mL) | | | | | | | | | | | | | | | |
| DSPC content | 51.5 | 45.9 | 50.9 | 53.3 | 56.5 | 51.3 | 59.1 | 67.3 | 48.0 | 76.1 | 71.4 | 61.8 | 57.6 | 44.2 | 44.6 |
| (%) | | | | | | | | | | | | | | | |
| Dry weight | 46.6 | 55.6 | 48.6 | 23.6 | 29.1 | 34.1 | 31.6 | 25.6 | 30.1 | 25.2 | 34.7 | 28.6 | 27.6 | 42.6 | 41.6 |
| (mg/mL) | | | | | | | | | | | | | | | |
| Molar | 4.6E+07 | 2.7E+09 | 1.1E+10 | 1.4E+08 | 5.9E+09 | 1.7E+10 | 4.2E+07 | 9.6E+09 | 1.6E+10 | 2.4E+08 | 1.3E+09 | 7.2E+08 | 2.1E+07 | 4.6E+09 | 1.9E+10 |
| absorption | | | | | | | | | | | | | | | |
| coefficient | | | | | | | | | | | | | | | |
| (λmax) | | | | | | | | | | | | | | | |
| | Sample name | | | | | | | | | | | | | | |
| | p6-1 | p6-2 | p6-3 | p7-1 | p7-2 | p7-3 | p8-1 | p8-2 | p8-3 | p9-1 | p9-2 | p9-3 | p10-1 | p10-2 | p10-3 |
| Kind of empty | Empty liposome 6 | | | Empty liposome 7 | | | Empty liposome 8 | | | Empty liposome 9 | | | Empty liposome 10 | | |
| liposome | | | | | | | | | | | | | | | |
| Internal | HEPES + dextran 130 mg/mL + | | | HEPES + NaCl 300 mM | | | HEPES + NaCl 300 mM | | | HEPES + dextran 130 mg/mL + | | | HEPES + dextran 130 mg/mL + | | |
| aqueous phase | NaCl 150 mM | | | | | | | | | NaCl 300 mM | | | NaCl 300 mM | | |
| External | HEPES + 0.308M sucrose | | | Citric acid + 0.308M | | | HEPES + 0.308M sucrose | | | Citric acid + 0.308M | | | HEPES + 0.308M sucrose | | |
| aqueous phase | | | | sucrose | | | | | | sucrose | | | | | |
| Concentration | 0.1 | 2 | 6 | 0.1 | 2 | 6 | 0.1 | 2 | 6 | 0.1 | 2 | 6 | 0.1 | 2 | 6 |
| of added ICG | | | | | | | | | | | | | | | |
| mg/ml | | | | | | | | | | | | | | | |
| OD (780 nm) | 5.4 | 14.1 | 23.1 | 5.8 | 95.2 | 252.0 | 5.1 | 22.4 | 21.4 | 4.9 | 115.2 | 372.0 | 8.2 | 73.1 | 60.8 |
| OD (895 nm) | 0.4 | 1.5 | 2.2 | 2.2 | 488.8 | 1522.8 | 0.5 | 3.8 | 1.4 | 0.9 | 479.2 | 2106.0 | 0.6 | 6.2 | 4.3 |
| 895/780 ratio | 0.08 | 0.11 | 0.09 | 0.39 | 5.13 | 6.04 | 0.10 | 0.17 | 0.06 | 0.18 | 4.16 | 5.66 | 0.07 | 0.09 | 0.07 |
| Maximum | 802 | 807 | 804 | 791 | 891 | 893 | 802 | 805 | 802 | 792 | 893 | 893 | 804 | 807 | 802 |
| absorption | | | | | | | | | | | | | | | |
| wavelength | | | | | | | | | | | | | | | |
| Particle size | 98 | 104 | 108 | 105 | 109 | 107 | 114 | 112 | 105 | 92 | 112 | 112 | 98 | 108 | 110 |
| (nm) | | | | | | | | | | | | | | | |
| Polydispersity | 0.175 | 0.124 | 0.092 | 0.251 | 0.067 | 0.118 | 0.238 | 0.176 | 0.131 | 0.142 | 0.100 | 0.148 | 0.169 | 0.175 | 0.189 |
| index (PDI) | | | | | | | | | | | | | | | |
| ICG | 0.03 | 0.09 | 0.16 | 0.02 | 0.86 | 2.40 | 0.03 | 0.15 | 0.15 | 0.04 | 1.00 | 3.63 | 0.05 | 0.18 | 0.42 |
| concentration | | | | | | | | | | | | | | | |
| (mg/mL) | | | | | | | | | | | | | | | |
| ICG content | 0.1 | 0.2 | 0.3 | 0.1 | 2.3 | 9.4 | 0.2 | 0.6 | 0.7 | 0.1 | 2.3 | 7.2 | 0.1 | 0.7 | 1.0 |
| (%) | | | | | | | | | | | | | | | |
| DSPC | 24 | 24 | 26 | 15 | 17 | 17 | 16 | 18 | 14 | 22 | 21 | 24 | 26 | 26 | 26 |
| concentration | | | | | | | | | | | | | | | |
| (mg/mL) | | | | | | | | | | | | | | | |
| DSPC content | 53.8 | 56.8 | 57.3 | 66.0 | 44.0 | 65.0 | 88.2 | 76.3 | 64.5 | 47.2 | 49.5 | 47.7 | 73.1 | 94.0 | 64.8 |
| (%) | | | | | | | | | | | | | | | |
| Dry weight | 44.2 | 42.2 | 46.2 | 22.6 | 38.1 | 25.6 | 18.7 | 24.2 | 22.2 | 45.6 | 42.6 | 50.6 | 36.2 | 27.2 | 40.2 |
| (mg/mL) | | | | | | | | | | | | | | | |
| Molar | 4.4E+07 | 1.4E+08 | 2.2E+08 | 9.5E+07 | 5.1E+09 | 2.2E+10 | 1.5E+08 | 4.6E+08 | 3.9E+08 | 2.7E+07 | 4.8E+09 | 1.8E+10 | 8.0E+07 | 1.2E+09 | 6.9E+08 |
| absorption | | | | | | | | | | | | | | | |
| coefficient | | | | | | | | | | | | | | | |
| (λmax) | | | | | | | | | | | | | | | |

### Example 5

Confirmation was made as to whether the ICG content was able to be additionally increased by increasing the addition amount of ICG at the time of the encapsulation treatment.

### Example 5-1

### Preparation of Empty Liposome

An empty liposome was prepared in the same manner as in Example 4-1 through the use of the dextran-added HEPES solution as the internal aqueous phase (solution A) and the sucrose-added citric acid buffer solution as the external aqueous phase (solution B), the solutions serving as conditions for the preparation of the empty liposome 2 in Example 4-1, provided that empty liposomes having different sizes, specifically, empty liposomes each having a particle size of 184 nm or 114 nm were prepared by changing the number of times of the extruder treatment.

### Example 5-2

### Encapsulation and Purification of ICG Utilizing pH Gradient

Indocyanine green was dissolved in the sucrose-added citric acid buffer solution as an external aqueous phase solution to produce an ICG solution having an ICG concentration of 6 mg/ml. The ICG solution was added to each of the two kinds of empty liposome dispersion liquids, and then the same operations as those of Example 4-2 were performed to prepare a p11-1 and a p12-1, provided that no extruder treatment was performed. In addition, after the purification and concentration treatments, a treatment with a 0.45-μm filter was performed.

In order to confirm whether the ICG content was able to be additionally increased, in the operations, the ICG solution (2.5 mL) was added to each liposome dispersion liquid (2.5 mL), and then the mixture was stirred at 60° C. for 30 minutes. After that, the ICG solution (2.5 mL) was added to the reaction liquid and then the mixture was stirred at 60° C. for an additional thirty minutes. The subsequent operations were performed in the same manner as in the foregoing to prepare a p11-2 and a p12-2.

### Example 5-3

### Absorption Spectrum Measurement

The maximum absorption wavelength of each of the four kinds of liposomes subjected to an ICG encapsulation treatment obtained in Example 5-2 was measured, and then its Abs895/Abs780 ratio was determined. Thus, the formation of the J-aggregate in the liposome was confirmed.

Table 17 shows the results. In each of all the samples, the Abs895/Abs780 ratio was 6 or more and hence the formation of the J-aggregate of ICG was able to be confirmed.

### Example 5-4

### Particle Size Measurement

The particle sizes of the four kinds of liposomes subjected to the ICG encapsulation treatment obtained in Example 5-2 were measured by DLS.

Table 17 shows the results. The particle size of each of the p11-1 and the p11-2 each having a large empty liposome particle size reduced after the encapsulation of ICG. On the other hand, the particle size of each of the p12-1 and the p12-2 each having a small empty liposome particle size increased after the encapsulation of ICG. The particle size of a liposome after the encapsulation of ICG changes from the particle size of an empty liposome and an additional investigation is needed for preparing a liposome having a desired particle size. However, it was found that the particle size of the liposome after the encapsulation of ICG was able to be changed depending substantially on the particle size of the empty liposome.

### Example 5-5

### Quantitative Determination of ICG, and Measurement of ICG Content and Molar Absorption Coefficient

The ICG concentration, ICG content, and molar absorption coefficient of each of the four kinds of liposomes subjected to the ICG encapsulation treatment obtained in Example 5-2 were measured in the same manner as in Example 4-5.

Table 17 shows the results. The ICG concentration, ICG content, or molar absorption coefficient of each of the p11-2 and the p12-2 to each of which a double amount of ICG had been added was able to be increased so as to be about twice as high as that of each of the p11-1 and the p12-1 to each of which a single amount of ICG had been added. It was able to be confirmed that the ICG content in the liposome was able to be additionally increased, and hence the increase of the addition amount was found to be effective for the preparation of a liposome type contrast agent for photoacoustic imaging having a high molar absorption coefficient.

### Example 5-6

### Quantitative Determination of DSPC

A DSPC content was calculated by performing the quantitative determination of DSPC in the same manner as in Example 4-6.

Table 17 shows the results. It was able to be confirmed that the ratio of DSPC to the particle weight was 30 wt % or more in each of all samples.

### Example 5-7

### Retention Ratio of ICG in Serum

The maximum absorption wavelength of ICG is 780 nm in the HEPES buffer solution and the maximum absorption wavelength changes to 800 nm in a fetal bovine serum (FBS). In addition, a difference between an absorbance in HEPES and that in the FBS becomes largest at 810 nm. The retention ratio of ICG in a liposome in a serum was evaluated by utilizing the fact that the difference between the absorbance at 810 nm in HEPES and that in the FBS enlarged according to the abundance ratio of free ICG.

Standard solutions of the HEPES buffer solution and the FBS in each of which a known concentration of ICG had been dissolved were produced, a difference in absorbance at 810 nm between the FBS standard solution and the HEPES standard solution was measured, and a standard curve was produced based on the ICG concentration and the difference in absorbance at 810 nm.

The ICG-encapsulating liposome (p11-1 or p12-1) was diluted with each of the HEPES buffer solution and the FBS so that an ICG concentration became 5 μg/ml. The diluted solutions were warmed for 24 hours at 37° C. under a light-shielding condition and then their absorbances at 810 nm were measured, followed by the determination of a difference between the absorbance of the ICG-encapsulating liposome at 810 nm in the FBS and that in the HEPES buffer solution. The difference in absorbance was converted into the amount of free ICG according to the standard curve and then the retention ratio of ICG in the liposome was determined.

Retention ratio (%) of ICG=concentration (μg/ml) of free ICG/5 μg/ml×100

Table 17 shows the results. Each of the liposomes had a retention ratio of ICG of 85% or more and hence ICG was found to be retained in the liposome even in the serum.

TABLE 17

| | Sample name | | | |
|---|---|---|---|---|
| | p11-1 | p11-2 | p12-1 | p12-2 |
| Empty liposome particle | 184 | | 114 | |
| size nm | | | | |
| Addition volume of ICG | 2.5 | 5 | 2.5 | 5 |
| solution ml | | | | |
| OD (780 nm) | 318 | 752 | 328 | 612 |
| OD (895 nm) | 2004 | 4632 | 2072 | 3916 |
| 895/780 ratio | 6.30 | 6.16 | 6.32 | 6.40 |
| Maximum absorption | 892 | 893 | 893 | 893 |
| wavelength | | | | |
| Particle size (nm) | 134 | 141 | 122 | 127 |
| Polydispersity index | 0.048 | 0.102 | 0.099 | 0.129 |
| (PDI) | | | | |
| ICG concentration | 3.71 | 7.72 | 3.56 | 6.92 |
| (mg/mL) | | | | |
| ICG content (%) | 8.0 | 15.0 | 10.3 | 16.3 |
| DSPC concentration | 28 | 33 | 28 | 25 |
| (mg/mL) | | | | |
| DSPC content (%) | 60.3 | 64.5 | 81.2 | 60.0 |
| Dry weight (mg/mL) | 46.5 | 51.5 | 34.5 | 42.5 |
| Molar absorption | 3.4E+10 | 7.8E+10 | 3.5E+10 | 6.3E+10 |
| coefficient (λmax) | | | | |
| Concentration of free | 0.72 | | 0.67 | |
| ICG in serum μg/ml | | | | |
| Retention ratio of ICG in | 86% | | 87% | |
| serum | | | | |

### Example 6

Confirmation was made as to whether an additionally small ICG-encapsulating liposome was able to be prepared by reducing the particle size of an empty liposome.

### Example 6-1

### Preparation of Small-Particle Size Empty Liposome

An empty liposome was prepared in the same manner as in Example 4-1 through the use of the dextran-added HEPES solution as the internal aqueous phase (solution A) and the sucrose-added citric acid buffer solution as the external aqueous phase (solution B), the solutions serving as conditions for the preparation of the empty liposome 2 in Example 4-1, provided that the following operations were performed instead of the extruder treatment. An empty liposome dispersion liquid was subjected to an ultrasonic dispersion treatment for 10 minutes with a probe type ultrasonic dispersing apparatus (UD-200, manufactured by TOMY SEIKO CO., LTD.) (irradiation level: 4) while being cooled in ice water. After that, the empty liposome dispersion liquid diluted with the HEPES buffer solution 4.2-fold was subjected to ultracentrifugation with an ultracentrifuge (himac CS150GXL, manufactured by Hitachi Koki Co., Ltd.) at 288,000 G for 17 minutes, and then the supernatant was recovered. The recovered small-particle size empty liposome had a particle size of 60 nm.

After that, the external aqueous phase of the small-particle size empty liposome dispersion liquid was replaced by ultrafiltration and then concentrated. Thus, the total lipid concentration of DSPC and cholesterol was adjusted to 4 mg/mL.

### Example 6-2

### Encapsulation and Purification of ICG Utilizing pH Gradient

Indocyanine green was dissolved in the sucrose-added citric acid buffer solution to produce an ICG solution having an ICG concentration of 6 mg/ml. 1.44 Milliliters of the ICG solution were added to 14.4 ml of the small-particle size empty liposome dispersion liquid, and then the same operations as those of Example 4-2 were performed to prepare a p13-1, provided that no extruder treatment was not performed and, after the purification and concentration treatments, a treatment with a 0.45-μm filter was performed.

### Example 6-3

### Absorption Spectrum Measurement

The maximum absorption wavelength of the p13-1 obtained in Example 6-2 was measured and then its Abs895/Abs780 ratio was determined. Thus, the formation of the J-aggregate in the liposome was confirmed.

Table 18 shows the results. The Abs895/Abs780 ratio was 4.59 and hence the formation of the J-aggregate of ICG was able to be confirmed.

### Example 6-4

### Particle Size Measurement

The particle size of the p13-1 obtained in Example 6-2 was measured by DLS.

As can be seen from the results shown in Table 18, the particle size became larger than the particle size of the empty liposome by 24 nm but the particle size of the liposome after the encapsulation of ICG was 84 nm. It was found that the use of the small-particle size empty liposome was able to reduce the particle size as compared with those of the J-aggregated ICG-encapsulating liposomes prepared heretofore. It was able to be confirmed that an additionally small particle size was preferred for improving tissue permeability in a body in a contrast agent and the preparation method enabled the preparation of an effective contrast agent.

### Example 6-5

### Quantitative Determination of ICG, and Measurement of ICG Content and Molar Absorption Coefficient

The ICG concentration, ICG content, and molar absorption coefficient of the p13-1 obtained in Example 6-2 were measured. Those results are as shown in Table 18.

### Example 6-6

### Quantitative Determination of DSPC

A DSPC content was calculated by performing the quantitative determination of DSPC in the same manner as in Example 4-6.

Table 18 shows the results. It was able to be confirmed that the ratio of DSPC to the particle weight was 30 wt % or more.

### Example 6-7

### Retention Ratio of ICG in Serum

The retention ratio of ICG of the p13-1 in a serum was determined in the same manner as in Example 5-7.

As shown in Table 18, the p13-1 had a retention ratio of ICG of 91% and hence it was able to be confirmed that the retention ratio of ICG in the serum was high.

TABLE 18

| | Sample name |
|---|---|
| | p13-1 |
| Empty liposome particle size nm | 60 |
| OD (780 nm) | 217 |
| OD (895 nm) | 997 |
| 895/780 ratio | 4.59 |
| Maximum absorption wavelength | 893 |
| Particle size (nm) | 84 |
| Polydispersity index (PDI) | 0.207 |
| ICG concentration (mg/mL) | 2.35 |
| ICG content (%) | 6.3 |
| DSPC concentration (mg/mL) | 27 |
| DSPC content (%) | 72.4 |
| Dry weight (mg/mL) | 37.2 |
| Molar absorption coefficient (λmax) | 5.0E+09 |
| Concentration of free ICG in serum | 0.43 |
| μg/ml | |
| Retention ratio of ICG in serum | 91% |

### Examples Corresponding to Embodiment 2

Specific reagents and reaction conditions to be used upon production of ICG-encapsulating lipid particles each showing a 700/780 ratio of 1 or more are given in the following examples. However, these reagents and reaction conditions can be modified, and such modifications are included in the scope of the present invention. Therefore, the following examples are intended to aid the understanding of the present invention and by no means limit the scope of the present invention.

As the reagents described below, there were used indocyanine green (ICG, manufactured by Society of Japanese Pharmacopoeia), distearoylphosphatidylcholine (DSPC, MC-8080, manufactured by NOF CORPORATION), a distearoylphosphatidylethanolamine-polyethylene glycol (DSPE-PEG, SUNBRIGHT DSPE-020CN, manufactured by NOF CORPORATION), cholesterol (manufactured by Wako Pure Chemical Industries, Ltd.), dextran 40 (manufactured by Tokyo Chemical Industry Co., Ltd.), dextran 70 (manufactured by Tokyo Chemical Industry Co., Ltd.), chloroform (KISHIDA CHEMICAL Co., Ltd., special grade), methanol (KISHIDA CHEMICAL Co., Ltd., special grade), and N-2-hydroxyethylpiperazine-N′-2-ethanesulfonic acid (HEPES, manufactured by DOJINDO LABORATORIES). It should be noted that in the dextrans used in the following examples, a dextran having a molecular weight of 40 kDa is the dextran 40, a dextran having a molecular weight of 70 kDa is the dextran 70.

### Example 7

### Comparative Example

### Preparation 1 of Lipid Particle Encapsulating ICG

61.2 Milligrams of DSPC, 20.4 mg of a DSPE-PEG, and 20.4 mg of cholesterol were dissolved in 1 mL of chloroform. Fifteen milligrams of ICG were dissolved in 1 mL of methanol. One milliliter of the chloroform solution and 1 mL of the methanol solution were charged into an eggplant flask and mixed, and then the solvents were distilled off at 40° C. under reduced pressure (Rotavapor R-205, manufactured by BUCHI), followed by the performance of vacuum drying (Vacuum oven VOS-301SD, manufactured by EYELA) overnight. 2.5 Milliliters of a dextran solution (whose dextran concentration was a concentration shown in Table 19) prepared by dissolving a dextran in a 10-mM HEPES solution (having a pH of 7.3) (hereinafter referred to as “HEPES solution”) were added to the resultant dried and hardened product of the lipids and ICG, and then the mixture was irradiated with an ultrasonic wave at 60° C. for 30 minutes (three-frequency ultrasonic cleaner VS-100III, AS ONE Corporation). Next, the mixture was subjected to an ultrasonic treatment with a probe type ultrasonic irradiation apparatus (Ultrasonic disruptor UD-200, manufactured by TOMY SEIKO CO., LTD.) (output level: 4) for 10 minutes in ice water. After that, 8 mL of the HEPES solution were added to the treated product, and then the mixture was subjected to ultracentrifugation at room temperature and 280,000×g for 17 minutes (manufactured by Hitachi Koki Co., Ltd., CS150GXL), followed by the recovery of the precipitate. Further, the centrifugal operation was repeated twice as described below. The precipitate was resuspended in 1 mL of the HEPES solution and then the suspension was subjected to ultracentrifugation (under the same conditions as those described above), followed by the recovery of the precipitate. The precipitate obtained through a total of three times of the ultracentrifugation was resuspended in 0.5 mL of the HEPES solution to prepare an ICG-encapsulating lipid particle. Seven kinds of ICG-encapsulating lipid particles were prepared by changing a dextran molecular weight and a dextran concentration as shown in Table 19. The name of each sample was also shown in Table 19.

It should be noted that the samples C0 and PLD1 were each prepared again in the same manner as in the foregoing, i.e., a total of two lots were prepared to confirm preparation repeatability.

TABLE 19

| | Sample name | | | | | | |
|---|---|---|---|---|---|---|---|
| | C0 | PLD1 | PLD2 | C1 | PLD3 | PLD4 | C2 |
| Dextran molecular | — | 40 | 40 | 40 | 70 | 70 | 70 |
| weight (KD) | | | | | | | |
| Dextran concentration | 0 | 130 | 26 | 5.2 | 130 | 26 | 5.2 |
| (mg/ml) | | | | | | | |

### Example 8

### Preparation 2 of Lipid Particle Encapsulating ICG

90 Milligrams of DSPC and 30 mg of cholesterol were dissolved in 1 mL of chloroform. Fifteen milligrams of ICG were dissolved in 1 mL of methanol. One milliliter of the chloroform solution and 1 mL of the methanol solution were charged into an eggplant flask and mixed, and then the solvents were distilled off at 40° C. under reduced pressure. Thus, a dried and hardened product of the lipids and ICG was obtained. The dried and hardened product of the lipids and ICG was dissolved in 1.6 mL of chloroform to prepare a chloroform solution of the lipids and ICG.

2.6 Grams of dextran 40 and 30 mg of a DSPE-PEG were dissolved in 20 mL of the HEPES solution to prepare a solution of the DSPE-PEG and the dextran. The total amount of the chloroform solution of the lipids and ICG was dropped to the solution, and then the mixture was subjected to an ultrasonic treatment with a probe type ultrasonic irradiation apparatus for 2 minutes while being cooled in ice water. Thus, an emulsion liquid was obtained. Chloroform was distilled off from the emulsion liquid with a rotary evaporator at 40° C. under reduced pressure for 2 hours. Nineteen milliliters of the liquid from which chloroform had been distilled off were subjected to solvent replacement with the HEPES solution by using an ultrafilter (Amicon Ultra-15, for a 100-KD fraction, manufactured by Millipore Corporation), and then 10 ml of the solution subjected to the solvent replacement were recovered. The solvent replacement was performed until the original liquid was diluted with the HEPES solution 34-fold. The solution subjected to the solvent replacement was subjected to ultracentrifugation three times in the same manner as in Example 7 to prepare an ICG-encapsulating lipid particle EPLD1.

In addition, an ICG-encapsulating lipid particle EPLD0 was prepared by using a DSPE-PEG solution to which dextran 40 was not added instead of the solution of the DSPE-PEG and the dextran. The EPLD0 was prepared by the same method as the method for the preparation of the EPLD1 except that the solvent replacement operation with the ultrafilter was omitted.

### Example 9

### Absorption Spectrum Measurement

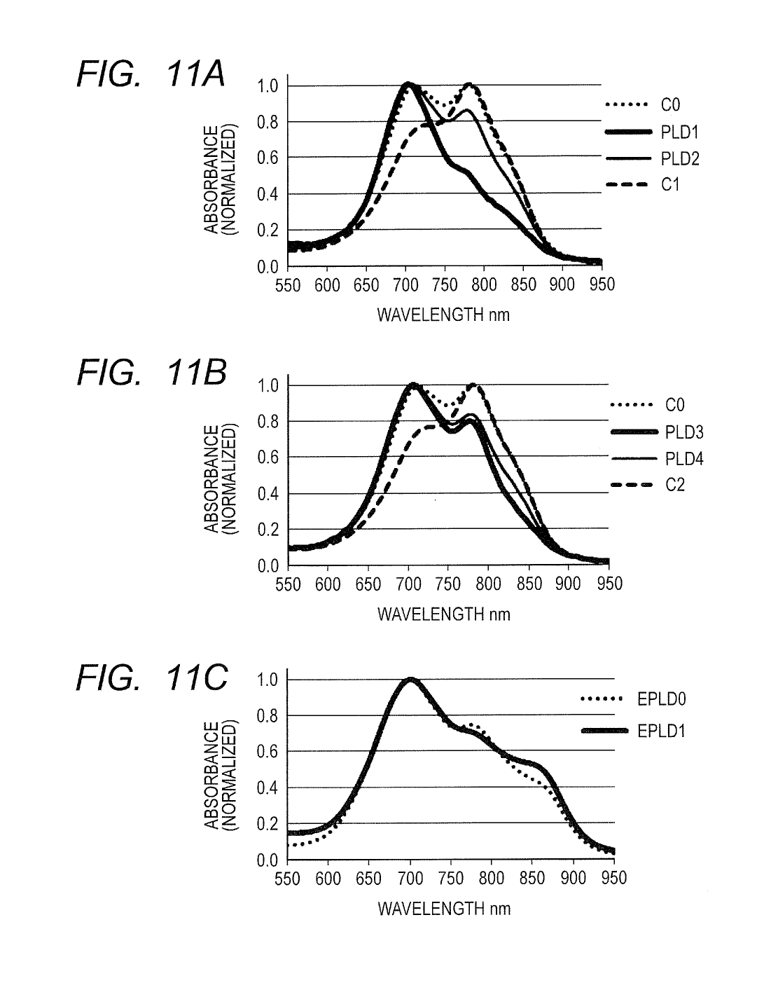

The absorption spectra of the nine kinds of ICG-encapsulating lipid particles obtained in Examples 7 and 8 were measured and shown in FIG. 11A, FIG. 11B, and FIG. 11C.

Absorption at 780 nm is derived from a monomer of ICG and that at 700 nm is derived from an H-aggregate. That is, a ratio of the absorbance at 700 nm to the absorbance at 780 nm (sometimes abbreviated as “700/780 ratio”) may represent the formation ratio of the H-aggregate. Table 20 shows the 700/780 ratio. It should be noted that in the measurement, a quartz cell having an optical path length of 1 cm was used and an aqueous solution prepared by properly diluting a lipid particle liquid with the HEPES solution was used.

### Example 10

### Particle Size of Lipid Particle

The particle sizes of the nine kinds (eleven samples) of ICG-encapsulating lipid particles obtained in Examples 7 and 8 were measured by the dynamic light scattering method (DLS method). As a result, all the particles were measured as monodisperse particles and their particle sizes based on cumulant analysis fell within the particle size range of 94 to 136 nm. Table 20 shows the particle sizes of the respective samples.

### Example 11

### Observation of Lipid Particle with Transmission Electron Microscope (TEM)

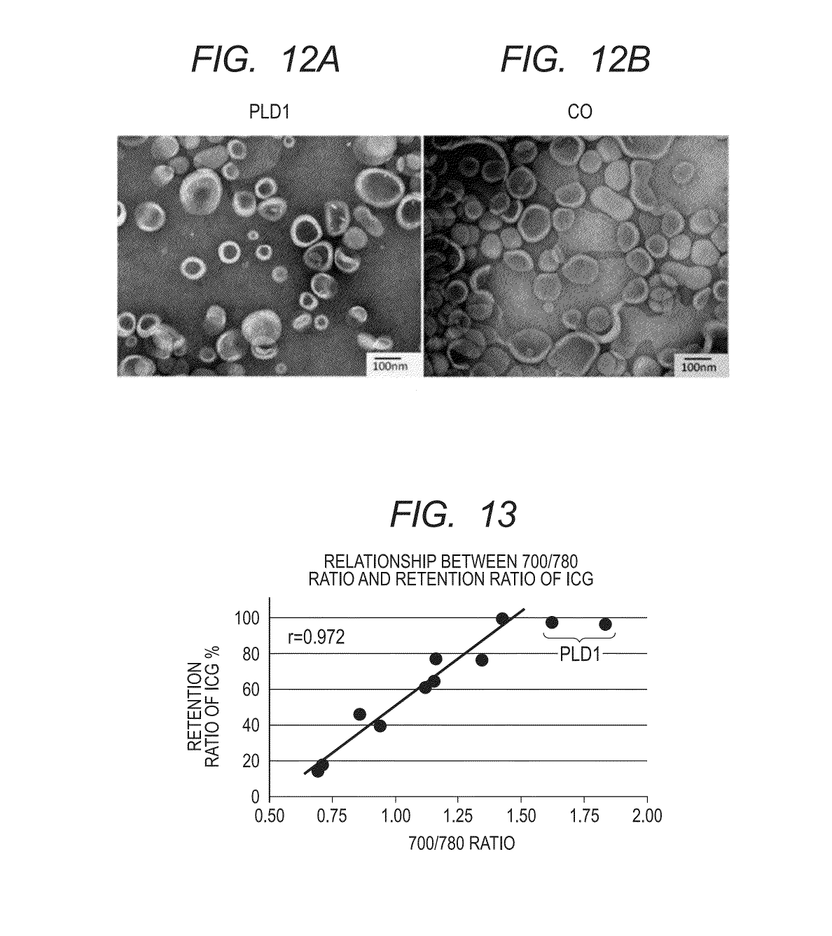

In order for the morphology of an ICG-encapsulating lipid particle to be confirmed, the samples PLD1 and C0 were each observed with a TEM. Each sample was stained by a negative staining method involving using a uranyl acetate stain and then observed with a TEM (H-7100FA, manufactured by Hitachi, Ltd.). FIG. 12A and FIG. 12B show the TEM images of the samples PLD1 and C0, respectively.

As a result of the observation, both the PLD1 and the C0 each showed typical liposome morphology. Each sample was mainly formed of a unilamellar liposome and a multilamellar liposome was also partly observed. The size of each sample is around 100 nm and may substantially coincide with the particle size measured by DLS.

### Example 12-1

### Evaluation for Retention Ratio of ICG in Particle in Serum

In order for each of the nine kinds (eleven samples) of ICG-encapsulating lipid particles obtained in Examples 7 and 8 to be measured for the retention ratio of ICG in the lipid particle in a living organism, a serum solution was used as an in vivo model. In other words, the retention ratio of ICG in the lipid particle in the serum was evaluated. A method for the evaluation is as described below.

Each sample was diluted with the HEPES solution, its absorbance was measured with a 96-well plate absorbance meter (VARIOS KAN, manufactured by Thermo Electron Corporation), and the concentration of each sample was adjusted so that the absorbance became 3 at the maximum absorption wavelength. Each of those samples was transferred to a sample tube and then diluted with a fetal bovine serum 10-fold to prepare a serum mixed liquid. The ICG concentration of the serum mixed liquid corresponds to a concentration of about 5 μg/mL and corresponds to a concentration in blood at the time of the in vivo fluorescent imaging of a mouse tumor to be described later.

Next, the serum mixed liquid was incubated in a dark place at 37° C. for 24 hours and then its light absorption spectrum in the wavelength range of 650 to 900 nm was measured with the 96-well plate absorbance meter. Next, 1 mL of the serum mixed liquid was subjected to ultracentrifugation (280,000 G, 17 minutes, room temperature) to precipitate the ICG-encapsulating lipid particle in the serum. Then, the light absorption spectrum of the centrifugation supernatant in the wavelength range of 650 to 900 nm was measured. It has been confirmed that ICG does not precipitate under the centrifugation conditions.

Therefore, the retention ratio of ICG in the lipid particle in the serum is defined as represented by the equation 1.

Retention ratio (%) of ICG=(1−integrated value of spectrum of centrifugation supernatant/integrated value of spectrum before centrifugation)×100 (Equation 1)

Table 20 shows the result of the retention ratio of ICG of each sample in the serum.

TABLE 20

| | Sample name | | | | | | | | | | |
|---|---|---|---|---|---|---|---|---|---|---|---|
| | C0 | | PLD1 | | PLD2 | C1 | PLD3 | PLD4 | C2 | EPLD0 | EPLD1 |
| Dextran | — | | 40 | | 40 | 40 | 70 | 70 | 70 | — | 40 |
| molecular | | | | | | | | | | | |
| weight KD | | | | | | | | | | | |
| Dextran | 0 | | 130 | | 26 | 5.2 | 130 | 26 | 5.2 | 0 | 130 |
| concentration | | | | | | | | | | | |
| at the time of | | | | | | | | | | | |
| preparation | | | | | | | | | | | |
| mg/ml | | | | | | | | | | | |
| Particle size | 106 | 105 | 132 | 136 | 109 | 94 | 129 | 109 | 102 | 116 | 116 |
| nm | | | | | | | | | | | |
| 700/780 ratio | 0.94 | 0.86 | 1.62 | 1.83 | 1.12 | 0.71 | 1.16 | 1.15 | 0.69 | 1.34 | 1.42 |
| Retention ratio | 39.1 | 45.8 | 97.6 | 96.3 | 60.7 | 17.9 | 77.0 | 64.8 | 14.4 | 76.7 | 99.2 |
| of ICG % | | | | | | | | | | | |

### Example 12-2

### Relationship Between 700/780 Ratio and Retention Ratio of ICG

FIG. 13 shows a relationship between the absorbance ratio 700/780 and retention ratio of ICG of each of the nine kinds (eleven samples) obtained in Examples 7 and 8. The coefficient of a correlation between the 700/780 ratio and the retention ratio of ICG was 0.972, and hence the ratios were found to show an extremely high positive correlation with each other, provided that upon calculation of the correlation coefficient, a numerical value for the retention ratio of ICG of the sample PLD1 reached saturation near a retention ratio of ICG of 100% and hence the correlation coefficient was determined after two pieces of data on the PLD1 had been excluded.

As shown in FIG. 13, the retention ratio of ICG in the serum was found to increase as the 700/780 ratio increased. It is apparent that the retention ratio of ICG in the serum can be increased as compared with that of a conventional ICG-encapsulating liposome (the C0 was described here as a conventional model) by preparing an ICG-encapsulating lipid particle having a 700/780 ratio of 1 or more as described above.

### Example 12-3

### Relationship Between Addition Concentration of Dextran and 700/780 Ratio

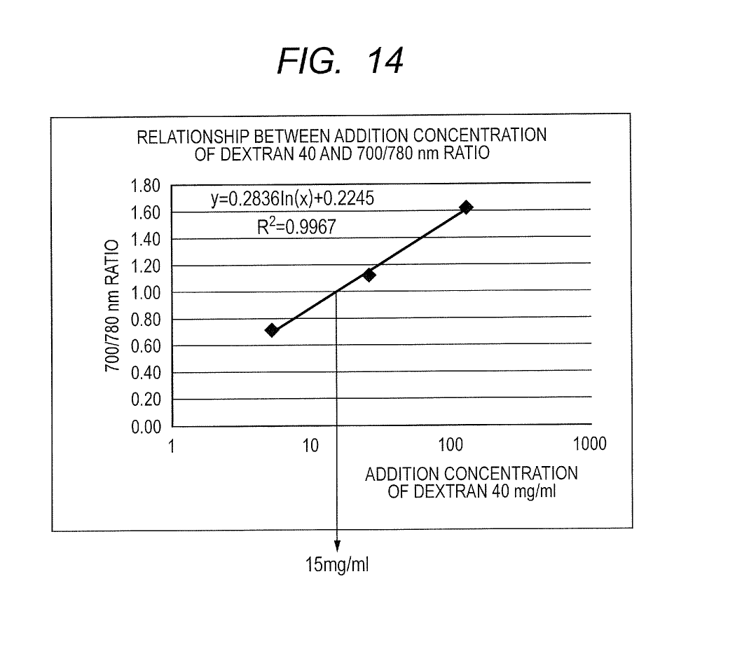

FIG. 14 shows a relationship between the addition concentration of dextran 40 and absorbance ratio 700/780 of each of the PLD1, PLD2, and C1 obtained in Example 7. The coefficient of a correlation between the addition concentration of dextran 40 and the absorbance ratio 700/780 was 0.997, and hence the concentration and the ratio were found to show an extremely high positive correlation with each other.

As shown in FIG. 14, the 700/780 ratio was found to increase as the addition concentration of dextran 40 increased. It is assumed from FIG. 14 that when the addition concentration of the dextran is 15 mg/ml (1.5 wt %) or more with respect to an aqueous medium, an ICG-encapsulating lipid particle having a 700/780 ratio of 1 or more can be prepared.

### Example 13

### Confirmation of Tumor-Contrasting Ability of Lipid Particle

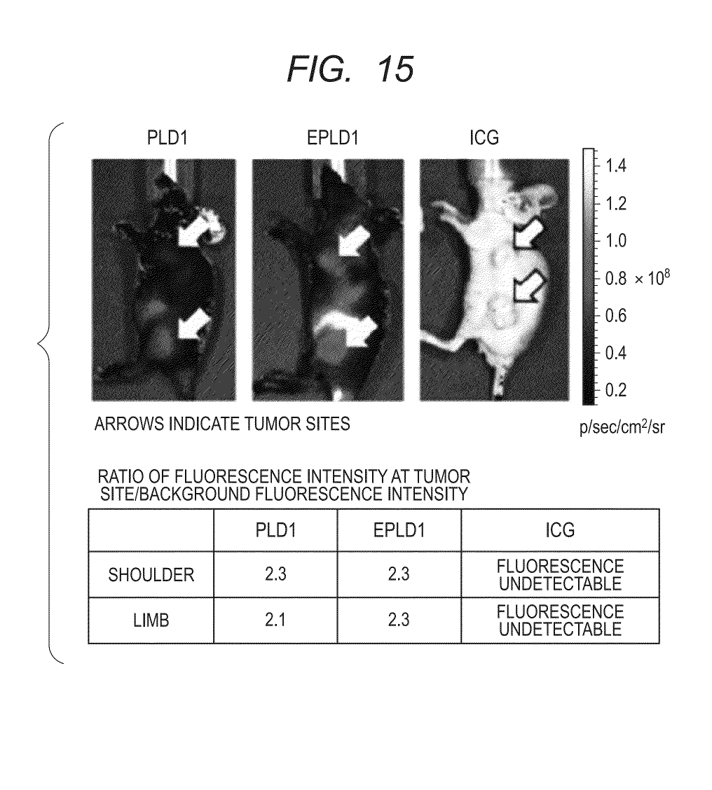

In order for each of the PLD1 and EPLD1 each showing a high retention ratio of ICG in the serum to be evaluated for its tumor-contrasting ability, each of both the samples was administered to a cancer-bearing mouse and then the fluorescent imaging of a tumor site thereof was performed. In the fluorescent imaging experiment, female outbred BALB/c Slc-nu/nu mice (six-week old at the time of purchase) (Japan SLC, Inc.) were used. About 2 weeks before the imaging experiment, 2×10⁶ N87 human stomach cancer cells (ATCC#CRL-5822) were subcutaneously injected into the shoulders and femurs of each mouse. The PLD1 and EPLD1 according to the present invention, and ICG as a control were administered to the mice, and then the three groups of mice were compared. The administration amount was 13 nmol (about 10 μg) per mouse in terms of a dye amount and the dye was injected as 100 μL of a HEPES solution into the tail vein of each mouse. With regard to the whole-body fluorescence image of the mouse to which a probe had been administered, the bright-field image and fluorescence image of the mouse were acquired with an IVIS (trademark) Imaging System 200 Series (XENOGEN) 24 hours after the administration. FIG. 15 is an image obtained by superimposing the bright-field image and fluorescence image of each mouse 24 hours after the administration. In each of the mice to which the PLD1 and the EPLD1 had been administered of FIG. 15, a fluorescence signal at a cancer-bearing site indicated by a white arrow was confirmed. A ratio (T/B ratio) of the fluorescence intensity of the tumor site to the background fluorescence intensity of a non-tumor site calculated for each of both the samples was 2.1 to 2.3, and hence it was able to be confirmed that the tumor site was able to be clearly contrasted. In addition, in the mouse to which ICG had been administered, a fluorescence signal at a cancer-bearing site indicated by a white arrow 24 hours after the administration could not be confirmed. The results showed that each of both the PLD1 and the EPLD1 was able to contrast a tumor, and showed its effectiveness as a tumor contrast agent.

### Example 14

### Particle Size Reduction Treatment

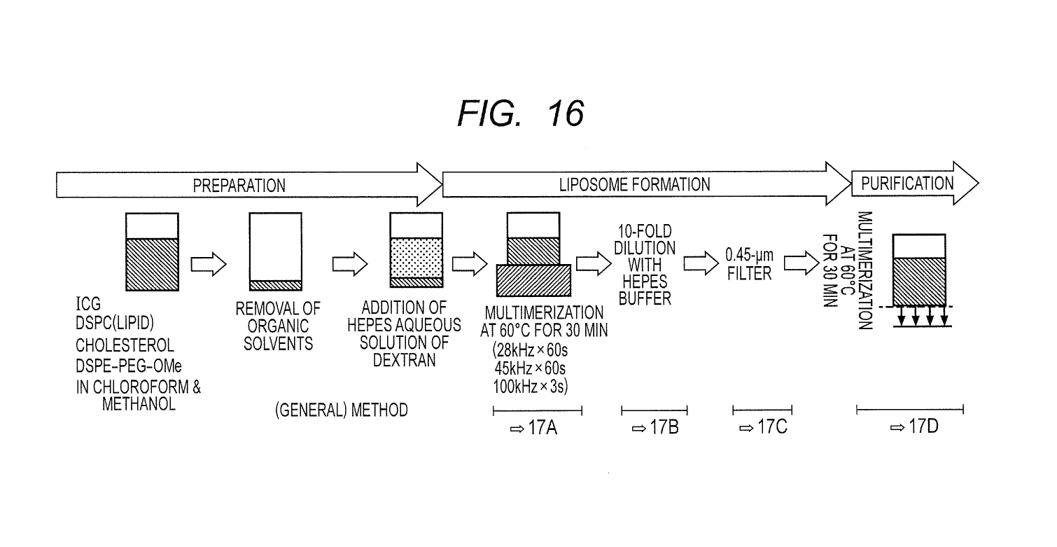

FIG. 16 illustrates the operation steps of this example. 61.2 Milligrams of DSPC, 20.4 mg of a DSPE-PEG, and 20.4 mg of cholesterol were dissolved in 1 mL of chloroform. Fifteen milligrams of ICG were dissolved in 1 mL of methanol. One milliliter of the chloroform solution and 1 mL of the methanol solution were charged into an eggplant flask and mixed, and then the solvents were distilled off at 40° C. under reduced pressure (Rotavapor R-205, manufactured by BUCHI), followed by the performance of vacuum drying (Vacuum oven VOS-301SD, manufactured by EYELA) overnight. 2.5 Milliliters of a dextran solution (whose dextran concentration was 130 mg/ml) prepared by dissolving a dextran in a 10-mM HEPES solution (having a pH of 7.3) were added to the resultant dried and hardened product of the lipids and ICG, and then the mixture was irradiated with an ultrasonic wave at 60° C. for 30 minutes (three-frequency ultrasonic cleaner VS-100III, AS ONE Corporation). Next, the mixture was subjected to an ultrasonic treatment with a probe type ultrasonic irradiation apparatus (Ultrasonic disruptor UD-200, manufactured by TOMY SEIKO CO., LTD.) (output level: 4) for 10 minutes in ice water. Hereinafter, a particle size is represented by a particle size distribution based on cumulant analysis measured by the dynamic light scattering method (DLS method).

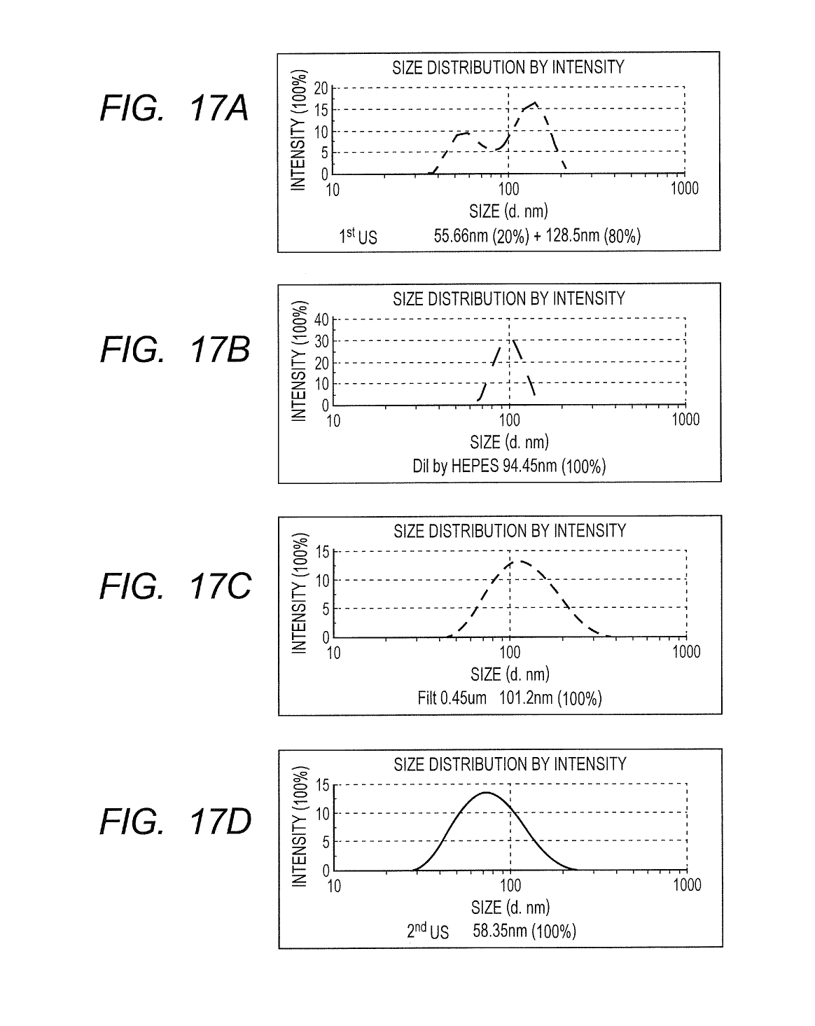

The liposome dispersion liquid thus obtained was subjected to a particle size reduction treatment by the following procedure. FIG. 17A, FIG. 17B, FIG. 17C, and FIG. 17D show particle size distributions before the treatment and after respective steps.

FIGS. 17A to 17D are each a particle size distribution diagram based on the cumulant analysis measured by the dynamic light scattering method (DLS method) in a main step of the particle size reduction treatment in Example 14 of the present invention. FIG. 17A is the particle size distribution diagram of an initial liposome dispersion liquid, FIG. 17B is the particle size distribution diagram of a liposome dispersion liquid prepared by diluting the initial liposome dispersion liquid with the HEPES solution (having a pH of 7.3) 10-fold, FIG. 17C is the particle size distribution diagram of a liposome dispersion liquid prepared by filtering the liposome dispersion liquid, which has been diluted with the HEPES solution (having a pH of 7.3) 10-fold, with a filter having a pore size of 0.45 μm, and FIG. 17D is the particle size distribution diagram of a liposome dispersion liquid after redispersion with an ultrasonic wave.

2.5 Milliliters of the initial liposome dispersion liquid (FIG. 17A, peaks of a mean particle size: 55.6 nm and 128.5 nm) were dropped to 22.5 ml of the 10-mM HEPES solution (having a pH of 7.3) to be diluted 10-fold (FIG. 17B, peak of a mean particle size: 94.45 nm).

Subsequently, the diluted liquid was filtered with a Minisart filter (pore size: 0.45 μm, size: 26 mm, membrane material: CA) manufactured by Sartorius Mechatronics Japan (FIG. 17C, peak of a mean particle size: 101.2 nm).

The filtrate was dispersed into 10 eggplant flasks in amounts of 2.5 ml each and then irradiated with an ultrasonic wave at 60° C. (three-frequency ultrasonic cleaner VS-1001II, AS ONE Corporation) for 30 minutes (FIG. 17D, peak of a mean particle size: 58.35 nm).

### Example 15

### Repeatability of Particle Size Reduction Treatment

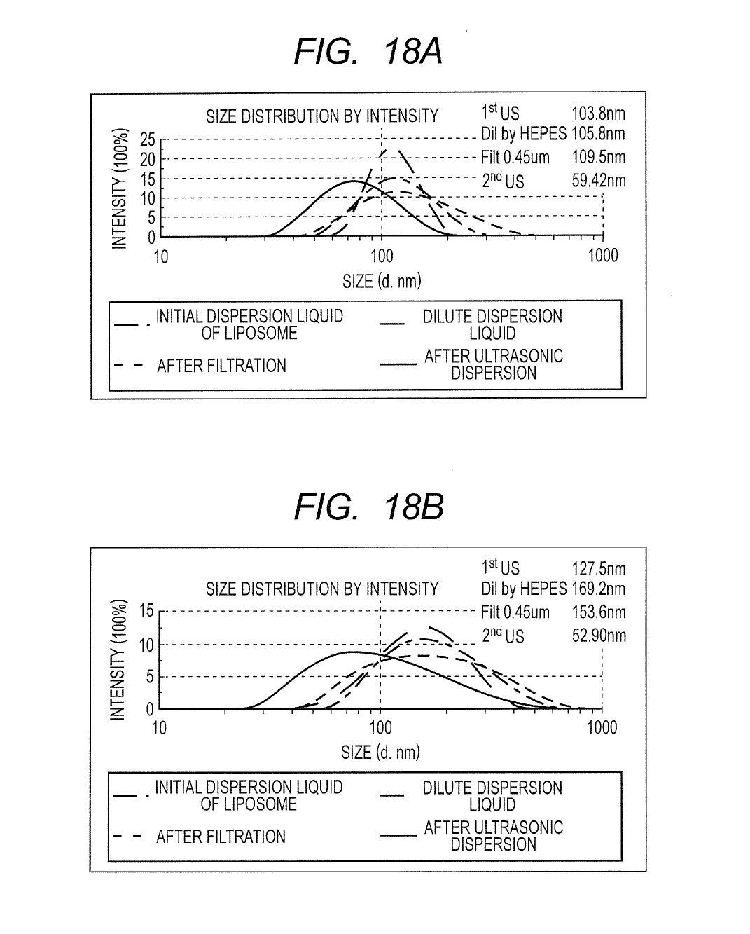

The same treatment as that of Example 14 was performed in another lot and each lot was subjected to the particle size reduction treatment of the present invention. FIGS. 18A and 18B each show particle size distributions before the treatment and after respective steps organized into 1 particle size distribution diagram (FIG. 18A and FIG. 18B).

In the lot shown in FIG. 18A, the mean particle size of the initial liposome dispersion liquid was 103.8 nm but the mean particle size was reduced to 59.42 nm by the particle size reduction treatment of the present invention. In the lot shown in FIG. 18B, the mean particle size of the initial liposome dispersion liquid was 127.5 nm but the mean particle size was reduced to 52.90 nm by the particle size reduction treatment of the present invention.

As can be seen from Examples 14 and 15, the mean particle size was 100 nm or more before the particle size reduction treatment, but a small-particle size liposome whose mean particle size converged in the range of 50 nm to 60 nm with good repeatability was obtained by the particle size reduction treatment of the present invention.

FIG. 18A and FIG. 18B are each a particle size distribution diagram based on the cumulant analysis measured by the dynamic light scattering method (DLS method) in a main step of the particle size reduction treatment in Example 15 of the present invention. FIG. 18A and FIG. 18B are particle size distribution diagrams for the liposome dispersion liquids of the lots different from each other, the particle size distribution diagrams being each obtained by organizing the particle size distributions of an initial liposome dispersion liquid, a liposome dispersion liquid after 10-fold dilution with the HEPES solution (having a pH of 7.3), a liposome dispersion liquid after filtration with a filter having a pore size of 0.45 μm, and a liposome dispersion liquid after redispersion with an ultrasonic wave into 1 diagram.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-038034, filed Feb. 23, 2012, Japanese Patent Application No. 2012-139646, filed Jun. 21, 2012, and Japanese Patent Application No. 2012-263002, filed Nov. 30, 2012, which are hereby incorporated by reference herein in their entirety.

### REFERENCE SIGNS LIST

1 J-aggregate
2 phospholipid
3 liposome
4 surfactant
101 indocyanine green (H-aggregate)
102 indocyanine green (monomer)
103 membrane
104 internal aqueous phase

What is claimed is:

1. A particle, comprising:
a J-aggregate of indocyanine green (ICG); and
a lipid having a positively charged region.

2. The particle according to claim 1, wherein a ratio of an absorbance for light having a wavelength of 895 nm to an absorbance for light having a wavelength of 780 nm is 0.1 or more.

3. The particle according to claim 1, further comprising, on a surface thereof, a surfactant.

4. The particle according to claim 1, further comprising a targeting molecule that specifically binds to a target site.

5. The particle according to claim 1, wherein the lipid forms a bilayer membrane.

6. The particle according to claim 1, wherein a ratio of the lipid to the particle is 30 wt % or more.

7. The particle according to claim 1, further comprising, on a surface thereof, a polyethylene glycol chain.

8. A contrast agent for photoacoustic imaging, comprising:
the particle according to claim 1; and
a dispersion medium in which the particle is dispersed.

9. A particle, comprising:
a phospholipid;
cholesterol; and
indocyanine green,
wherein a ratio of an absorbance of the particle at 700 nm to an absorbance thereof at 780 nm is 1 or more.

10. The particle according to claim 9, further comprising a dextran.

11. The particle according to claim 9, wherein the phospholipid forms a bilayer membrane.

12. The particle according to claim 9, wherein the phospholipid comprises distearoyl phosphatidylcholine (DSPC).

13. The particle according to claim 1, further comprising a polyethylene glycol.

14. A contrast agent, comprising the particle according to claim 9;
wherein the contrast agent is used in photoacoustic imaging.

15. A method of producing the particle according to claim 9, the method comprising the steps of:
dissolving raw materials for the particle containing the phospholipid, cholesterol, and indocyanine green in an organic solvent; and
removing the organic solvent to dry and harden the solution, followed by dispersion of the raw materials for the particle in an aqueous medium,
wherein a solution prepared by dissolving, in the aqueous medium, 1.5 wt % or more of a dextran with respect to the aqueous medium is used.

16. A method of producing the particle according to claim 9, the method comprising the steps of:
dissolving raw materials for the particle containing the phospholipid, cholesterol, and indocyanine green in an organic solvent; and
dropping the organic solvent in which the raw materials for the particle are dissolved to an aqueous medium,
wherein a solution prepared by dissolving a dextran in the aqueous medium is used.

17. The method according to claim 15, wherein the aqueous medium in which 2.6 wt % or more of the dextran is dissolved is used.

18. A method of producing a particle comprising treating the particle according to claim 9 through the steps of:
diluting the particle with a neutral aqueous buffer solution;
filtering the resultant with a filter having a pore having a pore size of 1 μm or less; and
dispersing the filtrate with an ultrasonic wave.

19. A particle, comprising:
an aggregate of indocyanine green; and
a phospholipid.

20. A contrast agent for photoacoustic imaging, comprising:
the particle according to claim 19; and
a dispersion medium.
